(19) United States
(12) Patent Application Publication
(10) Pub. No.: US 2016/0106680 A1
(43) Pub. Date: Apr. 21, 2016

(54) Abuse Deterrent Immediate Release Formulation

(71) Applicant: PHARMASCIENCE INC., Montréal (CA)

(72) Inventors: Krishna Hari Bhandari, Kirkland (CA); Naresh Talwar, Kirkland (CA)

(73) Assignee: Pharmascience Inc., Montréal, QC (CA)

(21) Appl. No.: 14/894,213

(22) PCT Filed: May 30, 2014
(86) PCT No.: PCT/CA2014/050506

§ 371 Date: Nov. 25, 2015

(30) Foreign Application Priority Data
May 31, 2013 (CA) 2817728

(51) Int. Cl. A61K 9/20; A61K 31/437; A61K 31/4458; A61K 31/4985; A61K 31/4468; A61K 31/5517; A61K 31/5513; A61K 31/485; A61K 31/135

(52) U.S. Cl. CPC A61K 9/2054; A61K 31/485; A61K 31/437; A61K 31/4458; A61K 31/135; A61K 31/4468; A61K 31/5517; A61K 31/5513; A61K 31/4985; A61K 9/205

(57) ABSTRACT

The present invention relates to an immediate release orally administrable abuse-deterrent pharmaceutical formulation comprising: at least one pharmaceutically active ingredient susceptible to abuse; at least one gelling polymeric compound selected from the group consisting of: polysaccharides, sugars, sugar derived alcohols, starches, starch derivatives, cellulose derivatives, Carrageenan, pectin, sodium alginate, gellan gum, xanthan gum, poloxamer, Carbopol®, PolyOx®, povidone, hydroxypropyl methylcellulose (HPMC), hypermellose, and combinations thereof; at least one disintegrant and optionally at least one surfactant, wherein said formulation exhibit properties related to deterring the abuse, via injection or nasal inhalation when being tampered and exposed to aqueous, alcoholic, acidic and basic media.

### FIELD OF THE INVENTION

The invention relates to an abuse deterrent immediate release oral formulations. More specifically, this invention relates to immediate release formulation containing pharmaceutically active ingredient susceptible to abuse, at least one gelling polymeric compound, wherein said formulation exhibit properties related to deterring the abuse, misuse, tampering, via injection or nasal inhalation of opioids of usual therapeutically effective dose.

### BACKGROUND OF THE INVENTION

The oral route remains the most desirable route for the administration of therapeutic agents because the low cost of therapy, manufacturing and ease of administration which lead to high levels of patient compliance.

Oral formulations for immediate release drug delivery system are a conventional type of drug delivery system and are designed to disintegrate and release their pharmaceutically active ingredient with no rate controlling features such as special coatings or other techniques.

An important goal of analgesic therapy is to achieve a continuous relief of pain. Regular administration of an analgesic is generally required to ensure that the next dose is given before the effects of the previous dose have worn off. Continuous suppression of pain through the use of around-the-clock opioid analgesics is now recommended in the treatment guidelines (Principles of Analgesic Use in the Treatment of Acute Pain and Cancer Pain, Fifth Ed., American, Pain Society (2003); Guideline for the Management of Cancer Pain in Adults, American Pain Society, 2005).

Generally, short-action opioids are considered appropriate for the treatment of transient pain types, such as acute, breakthrough, or chronic intermittent pain, which do not require long-lasting analgesia. Commonly prescribed Short-Acting Opioids (SAOs) include immediate-release (IR) morphine, hydromorphone, oxymorphone, codeine, fentanyl, hydrocodone, and oxycodone; codeine, hydrocodone, and oxycodone are also available in combination with acetaminophen or an Non-Steroidal Anti-Inflammatory Drug (NSAID), which limits the maximum daily dose because of the risk of liver and gastrointestinal toxic effects. [McCarberg B H, Barkin R L.].

When individuals start taking opioids, normally they are started on immediate release formulations and thereby require dosing every 4-6 hours in chronic pain. Opioids are common targets for both drug abusers and drug addicts. Most chronic pain patients need limit their intake of opioids to achieve a balance between the benefits of the drug and dose-limiting side effects.

When opioid-based prescription drugs are taken as directed by a physician for a short period of time, most patients will not develop a dependency for the product. However, similar to other opioids, misuse and abuse can easily lead to dependence and tolerance to opioids requiring more frequent and higher doses.

In the past 10 years, abuse of pharmaceutical opioids has significantly increased. Drug abusers and/or addicts typically may take a dosage form containing one or more opioid analgesics and crush, shear, grind, chew, dissolve and/or heat, extract or otherwise damage the product so that a significant amount or even an entire amount of the drug becomes available for immediate absorption by 1) injection, 2) inhalation, and/or 3) oral consumption.

In view of this, it is not surprising that the Food and Drug Administration's Division of Anesthetic, Analgesic and Rheumatology Drug Products and the U. S. Drug Enforcement Administration have encouraged companies to develop wide ranging abuse deterrent strategies for opioids (FDA Perspectives on Opioid Risk Management. Opioid Risk Management Meeting, Tufts Healthcare Institute, Boston, Mar. 29, 2005).

The preparation of immediate release opioids is disclosed in the following patents: U.S. Pat. No. 6,806,294 (WO200021520/Euro Celtique); U.S. Pat. No. 6,589,960 (Purdue Pharma); CA2547334 (U.S. Pat. No. 7,510,726; U.S. Pat. No. 7,476,402/Acura Pharmaceuticals).

Abuse is an ongoing concern that many pharmaceutical companies have tried to address. The prior art describes several methods and compositions intended to minimize the abuse of an opioid containing formulation. Various technologies to prevent drug abuse have been developed.

One of the approaches consists of combining in the same pharmaceutical formulation, an opioid agonist and an antagonist agent which is “sequestered” in a form that prevents it from being released when the medicinal product is taken normally, for example described in following Bristol Myers's patents: U.S. Pat. No. 3,966,940; U.S. Pat. No. 3,773,955. The same approach is disclosed in the following patents: CA2400578, CA2400567 (U.S. Pat. No. 6,696,088); and U.S. Pat. No. 8,236,351.

U.S. Pat. No. 8,105,631 (Purdue Pharma) describes oral dosage forms comprising a combination of an opioid agonist and an opioid antagonist, the opioid antagonist being included in a ratio to the opioid agonist to provide a combination product which is analgesically effective when the combination is administered orally. Such opioid antagonists have substantially increased effect when taken directly into the blood stream. Thus, abusing the opioid by crushing the tablet, dissolving it, and injecting or snorting (intranasal administration), would cause the antagonist to have its full effect, essentially blocking the opioid receptors, preventing the abuser from receiving an opioid effect, and inducing withdrawal in opioid-dependent individuals.

Another concept to prevent abuse relies on Gruenenthal's employed approach based on mechanical properties as to safeguard dosage form against abuse, particularly the high breaking strength of these pharmaceutical dosage form, in addition to one or several tamper-prone agents and optional physiologically acceptable adjuvants, make them tamper-resistant and secure against misuse. In the context of such tamper-resistant pharmaceutical dosage forms, reference can be made to the following patents: CA2534925 (WO2005016313), CA2534932 (WO 2005016314), CA2551231 (WO2005063214), WO2006002883, CA2572491 (WO2006002884), CA2572352 (WO2006002886), CA2595979 (WO2006082097), CA2595954 (WO2006082099), CA2713128 (WO2009092601), and a few applications WO201317242, WO201317234, relates to a tamper-resistant tablet comprising a matrix material and a plurality of coated particulates which preferably provides under in vitro conditions immediate release of the pharmacologically active compound.

WO200827442 (Theraquest Biosciences) discloses an abuse deterrent oral pharmaceutical formulations of opioid agonists and method of use for preventing or minimizing the risk of abuse and/or toxicity due to opioid agonists and an aversive agent which is sequestered in the intact dosage form but being releasable upon tampering of said dosage form. The aversive agent when released upon tampering of said dosage form at least partially blocking the effect of the opioid agonist and/or at least partially blocking the effect of another abusable drug not included in the dosage form. In said patent the opioid agonist is in sustained release form.

US 20100092553 and US 2007224129 (Endo Pharmaceuticals) discloses solid multiparticulate oral pharmaceutical forms whose composition and structure make it possible to deter misuse. The microparticles have an extremely thick coating layer which assures the modified release of the drug and simultaneously imparts crushing resistance to the coated microparticles so as to avoid misuse. Another example US 20110135731 describes an approach in which a pharmaceutical dosage form including an opioid antagonist surrounded by a controlled release matrix and an opioid agonist in a surrounding matrix.

CA2663172/WO2008033523 (Cima Lab.) discloses a pharmaceutical composition that may include a granulate which may include at least one active pharmaceutical ingredient susceptible to abuse mixed with at least two materials, a first material that is substantially water insoluble and at least partially alcohol soluble and a second material that is substantially alcohol insoluble and at least partially water soluble, wherein the active pharmaceutical ingredient and the two materials are granulated in the presence of water and alcohol. The composition may also include a coating on the granulate exhibiting crush resistance which may have a material that is deposited on the granulate using an alcohol based solvent. The composition further comprises a second particle comprising a fat/wax.

CA2707204, CA2661573 (Purdue Pharma/WO200823261) discloses a tamper resistant oral extended release pharmaceutical dosage form comprising an opioid analgesic in extended release matrix formulation. Said composition comprising at least one active agent and at least one polyethylene oxide in the form of a tablet or multiparticulates. Also is disclosed processes of manufacture, use and methods of treatment.

Another method to deter abuse of pharmaceutical formulation is to include a gelling agent which is intended to make it much more difficult for an abuser to tamper the dosage form and subsequently inhale, inject, and/or swallow the API recovered from the tampered dosage form. Essentially, a gelling agent works when a dosage form is being dissolved for extraction of the drug by forming a gel when placed in a solvent. Once formed, the gel prevents the misuse of the drug because of the gel formation which, in turn, cannot be abused intranasal, orally or intravenously.

Acura Pharm has approached formulations to prevent abuse of opioid-containing IR dosage forms in a different manner through the use of gelling agents in a matrix; reference can be made to the following patents and applications: CA2547334, CA2588725, CA2647360. These formulations comprise a therapeutically effective amount of any opioid drug substance that can be subject to abuse combined with a gel forming polymer, a nasal mucosal irritant, a flushing agent and a emulsifier. Such a dosage form comprising a gel forming polymer one or more of: polyethylene oxide polyvinyl alcohol, hydroxypropyl methyl cellulose and carbomer. The FDA has approved a new tablet formulation of immediate-release oxycodone (Oxecta®—King Pharmaceuticals/Pfizer) for management of acute and chronic moderate to severe pain, which is disclosed in the following patents: CA2547334 (U.S. Pat. No. 7,981,439; U.S. Pat. No. 7,510,726; U.S. Pat. No. 7,476,402—Acura Pharm//Pfizer). In Oxecta® formulation is used a tamper-resistant technology designed to deter oxycodone abuse by injection or nasal snorting. Dissolving the crushed tablet in water converts it into a viscous gel mixture, making it difficult to inject. Crushing the tablet and inhaling it through the nose causes burning and irritation.

Other applications, WO201179248 and WO2011411414, disclose pharmaceutical IR compositions to deter misuse, abuse and diversion of pharmaceutical dosage units containing drugs susceptible to abuse with generation of high volume foam upon contact with a suitable media.

In January 2013, the FDA proposed guidelines for establishing clear standards for manufacturers who develop and market tamper and abuse-resistant opioid products while considering incentives for undertaking the research and development necessary to bring such products to market. The FDA needs to unequivocally require drug companies to ensure that generic opioids are tamper resistant. (Miller, State Attorneys General Call on FDA to Strengthen Efforts on Tamper-Resistant Painkillers//FDA information on opiod medications, 2013).

There is a need for tamper-resistant dosage forms that exhibit properties related to deterring the abuse, via injection or nasal inhalation being in immediate release form.

Accordingly there exists a need for improved methods and new pharmaceutical formulations of immediate release dosage forms of opioids to provide quickly adjustment until the pain is controlled and relief symptoms amenable to treatment with the abusable drug.

The present invention therefore, in turn mitigates or eliminates some of the drawbacks of prior art formulation by providing matrix-based immediate release abuse deterrent formulation and providing a more conventional manufacturing process by preparing matrix-based immediate release abuse deterrent pharmaceutical dosage forms, which is less time consuming, therapeutically effective and less expensive. Furthermore, abuse deterrent immediate release formulations of the prior art have not shown to be resistant to abuse when exposed to various media after crushing. The present invention provides an abuse-deterrent approach to prevent extraction from a wide range of media (acidic, basic and hydroalcoholic).

### SUMMARY OF THE INVENTION

It is an object of the present invention to provide an immediate release abuse deterrent pharmaceutical formulation that provides an immediate release of the pharmaceutically active ingredient susceptible to abuse and that has advantages with respect to abuse deterrence in comparison with standard IR formulations.

It is an object of the present invention to provide an immediate release orally administrable abuse-deterrent pharmaceutical formulation comprising: at least one pharmaceutically active ingredient susceptible to abuse, at least one gelling polymeric compound, at least one disintegrant and optionally at least one surfactant, wherein said formulation becomes an uninjectable and unsyringeable gel when exposed to aqueous, alcoholic, acidic and basic media upon tampering.

According to the present invention a gelling polymeric compound is selected from pharmaceutically acceptable substances that hydrate in an aqueous medium to form a gel. These include polysaccharides, sugars, sugar derived alcohols, starches, starch derivatives, cellulose derivatives, synthetic polymers (e.g. polyvinyl alcohols, vinyl alcohol copolymers and starch/acrylate copolymers; and mixtures and copolymers thereof) gums (e.g. polygalactomannan gums, polyglucomanan gums, etc.) alginates (e.g. sodium alginate), Carrageenan; particularly pectin, sodium alginate, gellan gum, xanthan gum, poloxamer, Carbopol®, polyox, konjac glucomannan, povidone, hydroxypropyl methylcellulose (HPMC), hypermellose, and combinations thereof.

An embodiment of the present invention includes an immediate release orally administrable abuse-deterrent pharmaceutical formulation comprising: at least one pharmaceutically active ingredient susceptible to abuse; at least one gelling polymeric compound selected from a natural gum, a polymer or combinations thereof, a disintegrant and optionally at least one surfactant, wherein said formulation exhibit properties related to deterring the abuse, via injection or nasal inhalation when being tampered. Such compounds have gelling qualities when placed in contact with various media which makes them interesting for use in various pharmaceutical formulations.

Preferably, the immediate release orally administrable abuse-deterrent pharmaceutical formulation comprising: at least one pharmaceutically active ingredient susceptible to abuse; at least one gelling polymeric compound selected from the group consisting of: gellan gum, xanthan gum, konjac glucomannan, carrageenan, Carbopol® and combination thereof; a disintegrant and optionally at least one surfactant, wherein said formulation exhibit properties related to deterring the abuse, via injection or nasal inhalation when being tampered. Such compounds have gelling qualities when placed in contact with various media which makes them interesting for use in various pharmaceutical formulations.

More preferably, the immediate release orally administrable abuse-deterrent pharmaceutical formulation comprising beside mentioned above ingredients, at least one other pharmaceutically acceptable excipient. More preferably, the gelling polymeric compound or a combination of gelling polymeric compounds is present in a matrix in an amount ranging from 1.0% w/w to 30% w/w. Even more preferably, the gelling polymeric compound or a combination of gelling polymeric compounds is present in an amount ranging from about 1.0% w/w to about 20% w/w. In an embodiment of the present invention the gelling polymeric compound or a combination of gelling polymeric compounds present in an amount less than or equal to 530% w/w and at least one disintegrant. In an embodiment of the present invention invention the gelling polymeric compound or a combination of gelling polymeric compounds are present in an amount less than or equal to 520% w/w. In another embodiment of the present invention the gelling polymeric compound or a combination of gelling polymeric compounds are present in an amount less than or equal to 514% w/w. In a preferred embodiment of the present invention invention the gelling polymeric compound or a combination of gelling polymeric compounds are present in a matrix.

Preferably, the immediate release orally administrable abuse-deterrent pharmaceutical formulation comprising:
a) at least one pharmaceutically active ingredient susceptible to abuse;
b) at least one gelling polymeric compound, selected from a group comprising: a natural gum, a polymer and a combination thereof,
c) at least one disintegrant, and
d) optionally, a surfactant, wherein said formulation provides release of the active pharmaceutical ingredient and has an in vitro dissolution profile that is equal to or greater than 80 percent of the drug dissolved in 30 minutes after administration. In a particular embodiment of the present invention the pharmaceutically active ingredient susceptible to abuse is present in a matrix.

Another aspect of the present invention is to provide an immediate release orally administrable abuse-deterrent pharmaceutical formulation comprising: at least one pharmaceutically active ingredient susceptible to abuse; at least one natural gum; and at least one disintegrant that becomes an uninjectable and unsyringeable gel when exposed to aqueous, alcoholic, acidic or basic media upon tampering.

Preferably, the active pharmaceutical ingredient is selected from the group consisting of: opioids and morphine derivatives; antidepressants; stimulants; hallucinogenics; hypnotics; tranquilizers and other drugs susceptible to abuse. More preferably, the active pharmaceutical ingredient is selected from the group consisting of: amphetamine, alprazolam, codeine, diazepam, fentanyl & analogs, hydrocodone, hydromorphone HCl, lorazepam, meperidine, morphine, methylphenidate, methadone, nitrazepam, oxycodone HCl, oxymorphone, propoxyphene, temazepam, tramadol, zolpidem, zopiclone and combination thereof. In a preferred embodiment of the present invention the pharmaceutically active ingredient susceptible to abuse is selected from the group consisting of hydromorphone, hydrocodone, oxycodone, methylphenidate, zolpidem and combinations thereof.

In a preferred embodiment, the object of the present invention is to provide an abuse deterrent immediate release oral formulation which comprises a nasal irritant for a purpose to deter abuse via nasal administration. If an abuser crushes the dosage form, the nasal irritant is exposed. The nasal irritant is meant to discourage inhalation of the crushed dosage form by inducing pain and/or irritation. More preferably, the nasal irritant is sodium lauryl sulfate. According to the present invention, the nasal irritant can deter abuse of said formulation when a potential abuser tampers with a dosage form of the present invention. Preferably, if an abuser crushes the dosage form, the nasal irritant is exposed. The nasal irritant discourages inhalation of the crushed dosage form by inducing pain and/or irritation. More preferably, the nasal irritant is selected from the group consisting of capsaicin, piperine, allyl isothiocyanante, sodium lauryl sulfate and combinations thereof and discourages inhalation (e.g., via snorting through the nose) by inducing pain and/or irritation.

Yet another object of the present invention is to provide an abuse deterrent immediate release formulation comprising at least one active ingredient, susceptible to abuse; a gelling polymeric compound, and at least one pharmaceutically acceptable excipient, wherein said formulation provides an immediate release of the pharmaceutically active ingredient and has an in vitro dissolution profile that is not less than 80 percent of the drug dissolved in 30 minutes after administration, as measured by appropriate methods such as USP type I and II dissolution apparatus.

Preferably, the immediate release orally administrable abuse-deterrent pharmaceutical formulation according to the present invention provides an in vitro dissolution profile that releases more than 75% of the active ingredient within 10 min after proper administration (i.e. intended administration or non-abusive administration). Also preferably, provides an in vitro dissolution profile that releases more than 75% of the active ingredient dissolved within 20 min after administration. More preferably, provides an in vitro dissolution profile that is equal to or greater than 80% of the active ingredient dissolved within 30 min.

A further object of the present invention is to use the immediate release orally administrable abuse-deterrent pharmaceutical formulation for the treatment of pain, depression, anxiety or sleep disorders, narcolepsy and Attention-Deficit/Hyperactivity Disorder (ADHD) in human, wherein said formulation comprises: a therapeutically effective amount of an active pharmaceutical ingredient susceptible to abuse, at least one gelling polymeric compound, at least one surfactant, and at least one other pharmaceutically acceptable excipient.

### BRIEF DESCRIPTION OF THE DRAWINGS

FIG. 1 is a photograph of the gelling test of a tablet of Oxecta® in 10 mL of various solvents (water, acid, basic) following light shaking 20 times. After 4 minutes, phase separation occurred with a liquid upper layer and a solid cake of insoluble ingredients at the bottom.

FIG. 2 is a photograph of the gelling test of Oxecta® tablets after crushing and placed in different solvents to demonstrate its syringeability, injectability and filtration. In all solvents (water, acidic, basic), the top layer was syringeable & injectable through insulin syringe needle. It was filterable through 5 micron syringe filter.

FIG. 3 is a photograph of the gelling test of Oxecta® tablets after crushing and being placed in 10 ml of ethanol to demonstrate their syringeability, injectability and filterability. The filtered top layer in all media (10%, 20% and 40% v/v ethanol) was syringeable and injectable.

FIG. 4 is a photograph of the gelling test of an Oxecta® tablet in 10 ml of ethanol media and light shaking 20 times. After 2 minutes, phase separation occurs with a liquid upper layer and a solid cake of insoluble ingredients at the bottom.

FIG. 5 is a photograph of a syringe containing the resulting gel of an Oxecta® tablet showing their syringeability, injectability and filtration. The top layer in the dissolved solution was syringeable and injectable through an insulin syringe needle (pictured). It was filterable through a 5 micron syringe filter.

FIG. 6 is a photograph of the gelling test of a formulation according to the present invention following dissolution in 10 ml of various solvents (aqueous, pH 4, pH 7.5 & pH 12) following light shaking 20 times. Initially, there is a thick viscous fluid gel which after 3-5 minutes turns into a soft solid mass. It was very viscous but flowable in pH 1.1.

FIG. 7 is a photograph of the resulting dispersion in the gelling test of a formulation according to the present invention after crushing and dispersion in 10 ml of various ethanol concentrations (10%, 20% and 40% ethanol) following light shaking. After 5 minutes, there is still no phase separation in the media, there is a uniform mixture in all media (10%, 20% and 40% v/v ethanol). This mixture was not syringeable, injectable or filtrable. When this mixture was loaded from the back of a syringe plunger and forced through an insulin syringe needle, the lock failed resulting in gel spillover. It cannot pass through such needles even with high applied force.

### DETAILED DESCRIPTION OF THE INVENTION

The present invention relates to an abuse-deterrent immediate release formulation for oral administration comprising: a pharmaceutically active ingredient susceptible to abuse, at least one gelling polymeric compound, preferably a konjac glucomannan within a matrix wherein said formulation exhibit properties related to deterring the abuse, misuse, tampering, via injection, nasal inhalation or overdose of opioids consumption prescribed for use in the treatment or pain.

The present invention discloses an immediate release pharmaceutical formulation for oral administration comprising an active pharmaceutical ingredient susceptible to abuse, at least one gelling polymeric compound, which provides an immediate release of the pharmaceutically active ingredient when the tablet is taken orally and becomes an uninjectable and unsyringeable gel when exposed to aqueous, alcoholic, acidic and basic media upon tampering.

The term “immediate release”, as referred to herein, is defined to mean oral pharmaceutical compositions which when administered releases the active ingredient within a small period of time, Oral formulations for immediate release drug delivery system is a conventional type of drug delivery system and are designed to disintegrate and release their pharmaceutically active ingredient with no rate controlling features such as special coatings or other techniques.

The term “active ingredient” refers to Active Pharmaceutical Ingredients (API) which are active chemicals used in the manufacturing of drugs. The active agent can be a therapeutic, a prophylactic, or a diagnostic agent. The term “drugs susceptible to abuse” or “active pharmaceutical ingredient susceptible to abuse” refers to psychoactive drugs and analgesics including but not limited to opioids and drugs that can cause psychological and/or physical dependence on the drug.

The term “tampered dosage form” is defined for purposes of the present invention to mean that the dosage form has been manipulated by mechanical, thermal, and/or chemical means with the intended goal of affecting the original physical integrity and properties of the commercially available dosage form. An example of tampering of a dosage form is when one attempts to extract the therapeutic agent a commercially available dosage form for availability for immediate release. Extraction of a therapeutic agent from a commercially available dosage form can also be done in order to render the therapeutic agent available to abuse by an alternate administration route, e.g., parenterally or nasally. The tampering can be done, e.g., by means of crushing, milling, shearing, grinding, chewing, dissolution in a solvent, heating or even through a combination of such acts.

According to the present invention the active pharmaceutical ingredient susceptible to abuse is selected from the group consisting of: opioids, amphetamines, anti-depressants, hallucinogenics, hypnotics and major tranquilizers. Examples of drugs susceptible to abuse include alfentanil, alprazolam, allylprodine, alphaprodine, amphetamine, anileridine, benzylmorphine, bezitramide, buprenorphine, butorphanol, clonitazene, codeine, cyclazocine, desomorphine, dextromoramide, dezocine, diampromide, diazepam, dihydrocodeine, dihydroetorphine, dihydromorphine, dimenoxadol, dimepheptanol, dimethylthiambutene, dioxaphetyl butyrate, dipipanone, eptazocine, ethoheptazine, ethylmethylthiambutene, ethylmorphine, etonitazene etorphine, fentanyl, heroin, hydrocodone, hydromorphone, hydroxypethidine, isomethadone, ketobemidone, levallorphan, levorphanol, lofentanil, levophenacylmorphan, lorazepam, meperidine, meptazinol, metazocine, methadone, methylphenidate, metopon, morphine, myrophine, nalbuphine, narceine, nicomorphine, nitrozepam, norlevorphanol, normethadone, nalorphine, normorphine, norpipanone, opium, oxycodone, oxymorphone, papavereturn, pentazocine, phenadoxone, phenomorphan, phenazocine, phenoperidine, piminodine, piritramide, propheptazine, promedol, properidine, propiram, propoxyphene, sufentanil, temazepam, tramadol, tilidine, zolpidem, zopiclone, pharmaceutically acceptable salts thereof and prodrugs thereof.

More preferably, the active ingredients susceptible to abuse include but are not limited to hydromorphone, oxycodone, amphetamine, methylphenidate, morphine, fentanyl, hydrocodone, alprazolam, diazepam, lorazepam, nitrazepam, temazepam, zopiclone and zolpidem. In a preferred embodiment of the present invention the pharmaceutically active ingredient susceptible to abuse is selected from the group consisting of hydromorphone, hydrocodone, oxycodone, methylphenidate, zolpidem and combinations thereof.

The terms “uninjectable” and “unsuitable for injection” are defined for purposes of the present invention to mean that one would have substantial difficulty manipulating the tampered dosage form with the goal of injecting it with the use of a syringe. The main reasons which would justify a tampered dosage form to be unsuitable for injection are the following: due to pain upon administration or difficulty of pulling the drug into the syringe and/or pushing the dosage form through a syringe. The viscosity of the tampered dosage form thus reduces the potential for abuse of the drug in the dosage form. In a preferred embodiment, the gelling polymeric compound selected from the group of: polysaccharides, sugars, sugar derived alcohols, starches, starch derivatives, cellulose derivatives, carrageenan, pectin, sodium alginate, gellan gum, xanthan gum, poloxamer, carbopol, polyox, konjac glucomannan, povidone, hydroxypropyl methylcellulose (HPMC), hypermellose, and combination thereof, is present in such an amount in the dosage form to prevent the full evaporation of the solvent to an aqueous mixture of the dosage form. This, in turn, prevents to concentrate the therapeutic agent, and instead, produces a gel mass unsuitable for injection.

In addition to the pharmaceutically active ingredient susceptible to abuse, the pharmaceutical formulation according to the present invention contains at least one gelling polymeric compound selected from the group consisting of a gum, a polymer or a combination thereof, at least one disintegrant and at least one pharmaceutically acceptable excipient. The polymers have been identified as providing a deterrent to abuse, misuse, tampering, via injection, nasal inhalation or overdose of opioids consumption of usual therapeutically effective dose, when the tablet is crushed and mixed with water or other solvents.

Upon tampering the formulation, the gelling polymeric compound provides a gel-like quality to the tampered dosage form which slows the absorption of the opioids such that an abuser is less unlikely to obtain a rapid “high” since immediate release of the therapeutic agent is avoided. In a preferred embodiment, when the dosage form is tampered and exposed to a small amount (e.g., less than about 10 ml) of solvent (e.g., water, hydroalcohols, acid, or alkali), the dosage form will be unsuitable for injection and/or inhalation. Upon the addition of various solvents, the tampered dosage form becomes thick and viscous, rendering it unsuitable for injection.

Preferably, the formulation of the present invention comprising at least one pharmaceutically active ingredient, and at least one gelling polymeric compound, such as a gum, a polymer within a matrix selected from the group consisting of: gellan gum, konjac glucomannan, xanthan gum, carrageenan, Carbopol®, hydroxypropyl methylcellulose (HPMC) and combinations thereof. Preferably, the total amount of the gelling polymeric compound or combination of compounds present in a matrix ranges from about 1% w/w to about 30% w/w. More preferably, the gelling polymeric compound is present in a matrix in an amount ranging from about 1 w/w to about 20% w/w based on the total weight of said formulation. In certain embodiments of the present invention, the gelling polymeric compound is konjac glucomannan and is present in an amount ranging from about 1.0% w/w to about 20% w/w. In a preferred embodiment, the gelling polymeric compound is xanthan gum and is present in an amount ranging from about 1.0% w/w to about 20% w/w. In a preferred embodiment, the gelling polymeric compound is gellan gum and is present in an amount ranging from about 1.0% w/w to about 20% w/w. In another preferred embodiment, the gelling polymeric compound is carrageenan and is present in an amount ranging from about 1.0% w/w to about 20% w/w. In yet another preferred embodiment, the gelling polymeric compound is Carbopol® and is present in an amount ranging from about 1% w/w to about 20% w/w. In a preferred embodiment, the gelling polymeric compound is HPMC and is present in an amount ranging from about 1% w/w to about 20% w/w. In a preferred embodiment these gelling polymeric compounds are used in combination with each other.

In an embodiment, the formulation of the present invention comprises a pharmaceutically active ingredient susceptible to abuse; a combination of at least two gelling polymeric compounds selected from the group consisting of gellan gum, konjac glucomannan, xanthan gum, carrageenan, Carbopol® (carbomers), hydroxypropyl methylcellulose (HPMC) and combinations thereof a disintegrant and optionally, a surfactant, wherein said formulation provides release of the active pharmaceutical ingredient and has an in vitro dissolution profile that is not less than 80 percent of the drug dissolved in 30 minutes after administration.

In an embodiment of the present invention the total amount on the combination of gelling polymeric compounds ranges from 1% w/w to 30% w/w. In another embodiment of the present invention the total amount of a combination of gelling polymeric compounds present in a matrix ranges from about 1% w/w to about 20% w/w. In an embodiment of the present invention, the total amount of the combination of gelling polymeric compounds present is less than or equal to 530% w/w. In an embodiment of the present invention invention the gelling polymeric compound or a combination of gelling polymeric compounds are present in an amount less than or equal to 520% w/w. In another embodiment of the present invention invention the gelling polymeric compound or a combination of gelling polymeric compounds are present in an amount less than or equal to ≦14% w/w. In a preferred embodiment of the present invention invention the gelling polymeric compound or a combination of gelling polymeric compounds are present in a matrix.

The combination of gelling polymeric compounds is selected so the formulation of the present invention forms an uninjectable and unsyringeable gel when exposed to an acidic, basic or hydroalcoholic media. The present invention includes the use of combinations of at least two gelling polymeric compounds wherein one of the compounds is konjac glucomannan and at least one additional compound is selected from the group consisting of gellan gum, xanthan gum, carrageenan, Carbopol® (carbomers), hydroxypropyl methylcellulose (HPMC) and combinations thereof. The konjac glucomannan to the second gelling polymeric compound or combination of compounds ranges from about 3:1 to 9:1. In an embodiment of the present invention the combination of gelling polymeric compounds present is selected form the group consisting of:
1. konjac glucomannan and xanthan;
2. konjac glucomannan and Carbopol®;
3. konjac glucomannan, HPMC and gellan;
4. konjac glucomannan, gellan and PolyOx®;
5. konjac glucomannan, PolyOx®+carrageenan and gellan; and
6. konjac glucomannan, HPMC, carrageenan and gellan.

In an embodiment of the present invention the particle size of the gelling polymeric compounds is selected to obtain an uninjectable and unsyringeable gel within about 30 seconds to about 5 minutes when tampered without delaying the release of the pharmaceutically active ingredient when used as prescribed.

There are a number of available konjac gums on the market. The grades vary depending on the glucomannan content and viscosity of the gum. For example, grades of konjac gums are available were the konjac glucomannan content is above 71%, above 74%, above 80%, above 83%, above 86% and above 90%. The viscosities between grades can vary from 6-8*10³ mPa·s to 15-18*10³ mPa·s.

In an embodiment of the present invention the disintegrant used can contribute to the compressibility, flowability and homogeneity of the formulation. Further, it can also minimize segregation and help to provide an immediate release profile to the formulation. In an embodiment of the present invention the disintegrant does not form a gel when exposed to an acidic, basic or hydroalcoholic media. Preferably, the disintegrant is selected from the group consisting of: crospovidone, sodium starch glycolate, sodium pregelatinized starch, modified corn starch and combinations thereof. More preferably, the disintegrant is crospovidone and is present in an amount ranging from about 2% w/w to about 20% w/w of the total composition. The invention includes embodiments wherein the crospovidone is present in amounts between 5-20% w/w. In other embodiments of the present invention the crospovidone is present in a total amount of less than or equal to 11% w/w.

In an embodiment of the present invention the combination of gelling polymeric compounds is konjac glucomannan; present in an amount between 5-20% w/w and at least one additional gelling polymer compound is present in amounts between 1-7% w/w; wherein the ratio of konjac glucomannan to additional gelling polymer compound is between 3:1 to 9:1; the disintegrant is crospovidone and is present in an amount between 2-20% w/w; and the pharmaceutically active ingredient susceptible to abuse is selected from the group consisting of hydromorphone, hydrocodone, oxycodone, methylphenidate, zolpidem and combinations thereof. In a preferred embodiment the additional gelling polymer compound is selected from the group consisting of gellan gum, xanthan gum, Carrageenan, Carbopol® (carbomers), hydroxypropyl methylcellulose (HPMC) and combinations thereof.

More preferably, the formulation of the present invention provides according to the intended use immediate release of the pharmacologically active ingredient when the tablet is taken orally and becomes an uninjectable and unsyringeable gel when exposed to aqueous alcoholic, acidic and basic media upon tampering. The present formulation when being abused can discourage the abuser from injecting the gel intravenously or intramuscularly by making it extremely difficult, if not impossible to transfer an amount of active ingredient into solution to a syringe for injection.

In addition to the active ingredient susceptible to abuse and gelling agents, the pharmaceutical formulation of the present invention may contain optionally a surfactant added as a nasal irritant in order to deter nasal abuse. Nasal irritants include compounds that are generally considered pharmaceutically inert, yet can induce irritation under improper administration. Such compounds include, but are not limited to surfactants. Preferably, a suitable surfactant is selected from the group of: sodium lauryl sulfate, poloxamer, the sorbitan monoesters and glyceryl monooleates and combinations thereof. More preferably, surfactant is sodium lauryl sulfate and is present in an amount ranging from about 0.1% w/w to about 10.0% w/w based on the total weight of said formulation.

In addition to the active ingredient susceptible to abuse and gelling agents, the pharmaceutical formulation of the present invention contains the pharmaceutically acceptable excipients added to the composition for a variety of purposes. At least one pharmaceutically acceptable excipient may be present in the formulation of the present invention, but not limited to: diluents, fillers, binders, lubricants, diluents, disintegrants, surfactants, foam forming agents and combinations thereof. As understood by a person skilled in the art, these excipients are conventional excipients which are well known in the pharmaceutical art.

Preferably, the filler is selected from the group consisting of: cellulose, dibasic calcium phosphate, calcium carbonate, sucrose, lactose, glucose, mannitol, sorbitol, maltol, pregelatinized starch, corn starch, potato starch and combinations thereof. More preferably, the filler is microcrystalline cellulose and is present in an amount ranging from about 30% w/w to about 85% w/w of the total composition.

Preferably, the lubricant is selected from the group consisting of: magnesium stearate, calcium stearate, zinc stearate, sodium stearate, stearic acid, aluminum stearate, glyceryl behenate, hydrogenated vegetable oil and combinations thereof. More preferably, the lubricant is magnesium stearate and is present in an amount ranging from about 0.1% w/w to about 2.0% w/w of the total composition.

In an embodiment of the present invention a tablet matrix formulation comprises a gelling polymer compound or a combination thereof to prevent abuse by getting an uninjectable and unsyringeable gel in water, hydroalcohols, acids and alkali and to prevent nasal abuse. The amount of a gelling polymeric compound, such as polysaccharides, sugars, sugar derived alcohols, starches, starch derivatives, cellulose derivatives, synthetic polymers (e.g. polyvinyl alcohols, vinyl alcohol copolymers and starch/acrylate copolymers; and mixtures and copolymers thereof) gums (e.g. polygalactomannan gums, polyglucomanan gums, etc.) alginates (e.g. sodium alginate), Carrageenan; particularly pectin, sodium alginate, gellan gum, xanthan gum, poloxamer, Carbopol®, polyox, konjac glucomannan, povidone, hydroxypropyl methylcellulose (HPMC), hypermellose, and combinations thereof; in said formulation may vary with tablet strength and active ingredient, ranging from about 1.0% w/w to about 30% w/w based on the total weight of said formulation. More preferably the gelling polymeric compounds combination ranges at least from about 1.0% w/w to about 20% w/w based on the total weight of said matrix formulation. The matrix may optionally contain a surfactant or nasal irritant, or foam forming agent to prevent drug abuse, but not enough to impact the intended use.

Dissolution is an essential part of pharmaceutical development of solid oral dosage forms. The media and conditions chosen in the studies depend on the required release characteristics of the intended product. For immediate release products the paddle (Apparatus 2, usually at 50 to 75 rpm) and basket (Apparatus 1, usually at 100 rpm) testing are the conventional method to determine dissolution rate. Immediate release typically means that 75% of the API is dissolved within 45 minutes. Lately, the terms rapidly dissolving (85% in 30 minutes) and very rapidly dissolving (85% in 15 minutes) have become popular and important in dissolution testing. The following media was considered for immediate release products during development studies: pH 6.8 buffer (or simulated intestinal fluid without enzymes); pH 4.5 buffer; pH 1.2 buffer (or simulated gastric fluid without enzymes) or 0.1 M hydrochloric acid; water may be considered as an additional medium. If both the test and reference product show more than 80% dissolution within 30 minutes, the profiles are considered similar.

In an embodiment of the present invention an immediate release pharmaceutical formulation comprising at least one active ingredient, susceptible to abuse; a gelling polymeric product, such as natural gums and polymers and optionally a nasal irritant or a foam forming agent and at least one pharmaceutically acceptable excipient, wherein said formulation provides an immediate release of the pharmaceutically active ingredient and has an in vitro dissolution profile that is equal to or greater than 75 percent of the drug dissolved in 10 minutes after administration, as measured by appropriate methods such as USP type I and II dissolution apparatus. Preferably, said formulation provides an immediate release of the pharmaceutically active ingredient and has an in vitro dissolution profile that is more than 75% of the active ingredient dissolved within 20 min. More preferably, said formulation provides an immediate release of the pharmaceutically active ingredient and has an in vitro dissolution profile that is more than 80% of the active ingredient dissolved within 30 min.

The following Examples illustrate the preferred embodiments but are not limiting the present invention.

### Example 1

### Tablet Preparation

To make an abuse-deterrent immediate release formulation of Hydromorphone HCl the following manufacturing steps were followed:

Step 1:

The required quantity of Hydromorphone HCl (8.0 mg) was mixed with required quantity of konjac glucomannan (10.0 mg). The required quantity of gellan gum (10.0 mg) was added to the mixed blend and required quantity of HPMC (10.0 mg) and was mixed thoroughly. Then, the required quantity of sodium lauryl sulfate (7.0 mg) or crospovidone XL (40.0 mg) were added and were mixed in a suitable blender thoroughly.

Step 2:

The obtained blend was mixed with ½ of the required quantity of microcrystalline cellulose (311.0 mg). The remaining ½ of the required quantity of microcrystalline cellulose was added and was mixed thoroughly. Then, the blend obtained was passed through a 40 mesh sieve.

Step 3:

The required quantity of magnesium stearate (4.0 mg) was mixed with 50 grams of blend from step 2 and passed through a 40 mesh sieve. The remaining mixture of step 2 was added and mixed for 30 seconds to 1 minute. Then, the blend obtained was direct compressed.

The formulation of Example 1 is set out in Table 1.

TABLE 1
Immediate release abuse-deterrent
formulation for direct compression.

Qty/Tab

| N | Ingredient | (mg) | % w/w |
|---|---|---|---|
| 1 | Hydromorphone HCl | 8.008 | 2.002 |
| 2 | Sodium lauryl sulfate | 7 | 1.75 |
| 3 | Gellan gum CG-HA | 10 | 2.5 |
| 4 | Konjac glucomannan | 10 | 2.5 |
| 5 | HPMC E10 | 10 | 2.5 |
| 6 | Crospovidone XL | 40 | 10 |
| 7 | Magnesium stearate | 4 | 1 |
| 8 | Microcrystalline | 311 | 77.748 |
| | cellulose pH 102 | | |
| | Total Core | 400 | 100 |

The tablets were monitored for weight, hardness, thickness and friability. The tablets were tested for assay, release characteristics (in vitro dissolution method) and abuse deterrent properties.

### Evaluation of Dissolution Profile

The pharmaceutical dosage form obtained from Example 1 was subsequently tested for in vitro dissolution rate, measured by Apparatus (USP Type II with paddle), using the following parameters:
Media: 500 ml of purified water
Speed: 50 rpm
Temperature: at 37° C.

The acceptable dissolution criterion is not less than 80% of the drug dissolved in 30 minutes. (U.S. Pharmacopoeia, XXVI, 2003)

The dissolution results are set out in Table 2.

TABLE 2
Dissolution rate of Hydromorphone abuse- deterrent
pharmaceutical formulation of Example 1.

| Time | Example 1/ | | | |
|---|---|---|---|---|
| (min) | Mean | Min | Max | % RSD |
| 10 | 86 | 81 | 88 | 3.5 |
| 15 | 89 | 83 | 91 | 3.0 |
| 20 | 90 | 86 | 91 | 2.3 |
| 30 | 89 | 85 | 91 | 2.5 |
| 45 | 89 | 85 | 90 | 2.1 |
| 60 | 89 | 86 | 90 | 1.9 |
| 75 | 89 | 85 | 91 | 2.4 |

Conclusion: an in vitro dissolution criterion of NLT 80% of the drug dissolved in 30 minutes was met.

### Evaluation of Gelation Behaviour

Each coated tablet was crushed in a mortar and pestle to get fine powder. This was then transferred to 20 mL clear glass vial and 10 mL of solution media was added. It was stirred immediately vigorously and the time taken to get a mass that did not fall while inverting the bottle was noted.

The gel time was measured using media at room temperature as well as using boiling media and further boiling the mixture.

### Example 2

### Tablet Preparation

To make an abuse-deterrent immediate release formulation of Hydromorphone the following manufacturing steps were followed:

Step 1:

The required quantity of Hydromorphone HCl (8.0 mg) was mixed with required quantity of konjac glucomannan (5.0 mg). Same as example 1. The required quantity of gellan gum (5.0 mg) was added to the mixed blend and required quantity of HPMC (5.0 mg) and also was mixed thoroughly. Then, the required quantity of sodium lauryl sulfate (7.0 mg) and the required quantity of crospovidone XL (40.0 mg) were added and were mixed thoroughly.

Step 2:

The obtained blend from previous step was mixed with ½ of the required quantity of microcrystalline cellulose (326.0 mg). The remaining ½ of the required quantity of microcrystalline cellulose was added and was mixed thoroughly. Then, the blend obtained was passed through a 40 mesh sieve.

Step 3:

The required quantity of magnesium stearate (4.0 mg) was mixed with 50 grams of blend from step 2 and passed through a 40 mesh sieve. The remaining mixture of step 2 was added and mixed for 30 seconds to 1 minute. Then, the blend obtained was compressed.

The formulation of Example 2 is set out in Table 3.

TABLE 3
Abuse-deterrent immediate release formulation
of Hydromorphone of Example 2.

Qty/Tab

| N | Ingredient | (mg) | % w/w |
|---|---|---|---|
| 1 | Hydromorphone HCl | 8.0 | 2.0 |
| 2 | Sodium lauryl sulfate | 7.0 | 1.75 |
| 3 | Gellan gum CG-HA | 5.0 | 1.25 |
| 4 | Konjac glucomannan | 5.0 | 1.25 |
| 5 | HPMC E10 | 5.0 | 1.25 |
| 6 | Crospovidone XL | 40.0 | 10.0 |
| 7 | Magnesium stearate | 4.0 | 1.0 |
| 8 | Microcrystalline | 311.0 | 81.49 |
| | cellulose pH 102 | | |
| | Total Core | 400 | 100 |

The tablets were monitored for weight, hardness, thickness and friability. The tablets were tested for assay, release characteristics (in vitro dissolution method) and abuse deterrent properties.

### Evaluation of Dissolution Profile

The pharmaceutical dosage form obtained from Example 2 was subsequently tested for in vitro dissolution rate, measured by Apparatus (USP Type II with paddle), using the following parameters:
Media: 500 ml of purified water
Speed: 50 rpm
Temperature: at 37° C.

The acceptable dissolution criterion is not less than 80% of the drug dissolved in 30 minutes. (U.S. Pharmacopoeia, XXVI, 2003).

The dissolution results are set out in Table 4.

TABLE 4
Dissolution rate of Hydromorphone abuse- deterrent
pharmaceutical formulation of Example 2.

| Time | Example 3/ | | | |
|---|---|---|---|---|
| (min) | Mean | Min | Max | % RSD |
| 10 | 85 | 84 | 89 | 2.6 |
| 15 | 87 | 85 | 89 | 1.8 |
| 20 | 89 | 86 | 91 | 1.7 |
| 30 | 89 | 87 | 90 | 1.4 |
| 45 | 90 | 88 | 92 | 1.4 |
| 60 | 91 | 89 | 92 | 1.5 |
| 75 | 91 | 89 | 93 | 1.8 |

Conclusion: an in vitro dissolution criterion of NLT 80% of the drug dissolved in 30 minutes was met.

### Evaluation of Gelation Behaviour

Each tablet was crushed in a mortar and pestle to get fine powder. This was then transferred to 20 mL clear glass vial and 10 mL of solution media was added. It was stirred immediately vigorously and the time taken to get a mass that did not fall while inverting the bottle was noted.

In order to assess the effectiveness of the developed formulation to deter potential abusers from extracting an opioid substance (hydromorphone) from an immediate release formulation, tests were carried out to determine the time to gellation of a crushed tablet of tablets from Example 2 in 10 ml of various media at room temperature.

### Syringeability and Injectability

In order to abuse the drugs via injection route, abusers typically crush the tablet and dissolve in small amount of water to extract the soluble drug. The ease in the drawing of the mass into the syringe (syringeability) and injection of the mass in the syringe (injectability) was determined using the insulin syringe which they typically use. Crushed tablets of Example 2 quickly turned into a solid gel-like mass within a few minutes in the cold media and within a minute in the hot media that did not fall upon inversion of the vial within few minutes in cold media and within a minute in hot media. Due to such solid-like consistency of crushed tablets in those media (Table 10), it was not possible to draw the mass into a syringe for subsequent injection.

In order to assess the effectiveness of present formulation to deter potential abusers from extracting an opioid substance from an immediate release formulation, gelation tests were carried out to determine the time to gelation of a crushed tablet from Example 2 in 10 ml of media at room temperature (Table 5). Time taken to get a mass that did not fall while inverting the glass vial at 180° was noted. The faster this solid mass is formed, the lower are the chances of drawing the solution and injecting by potential abusers is. It was noted that the gelation time was within 3 minutes in non-alcoholic media covering almost the whole pH ranges. Since abusers typically try to dissolve the drug in water, the quick gelation time in water was of added value. Similarly, the tablet formula gelled at 10% ethanol in water. In higher ethanol concentration, a thick liquid viscous fluid mass was obtained. However, in those cases, the viscous fluid mass fell while inverting the glass vial to 180°.

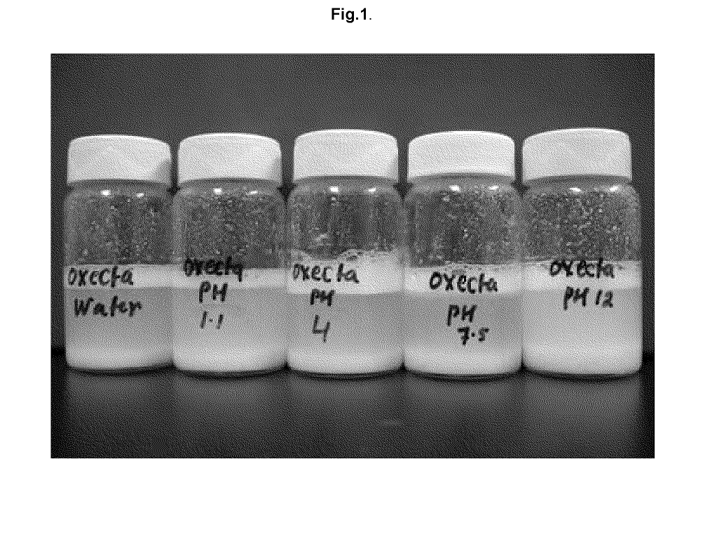

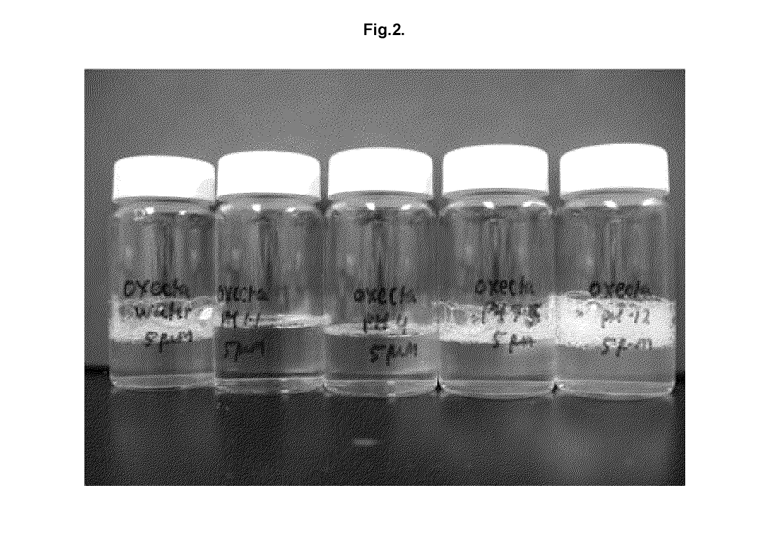

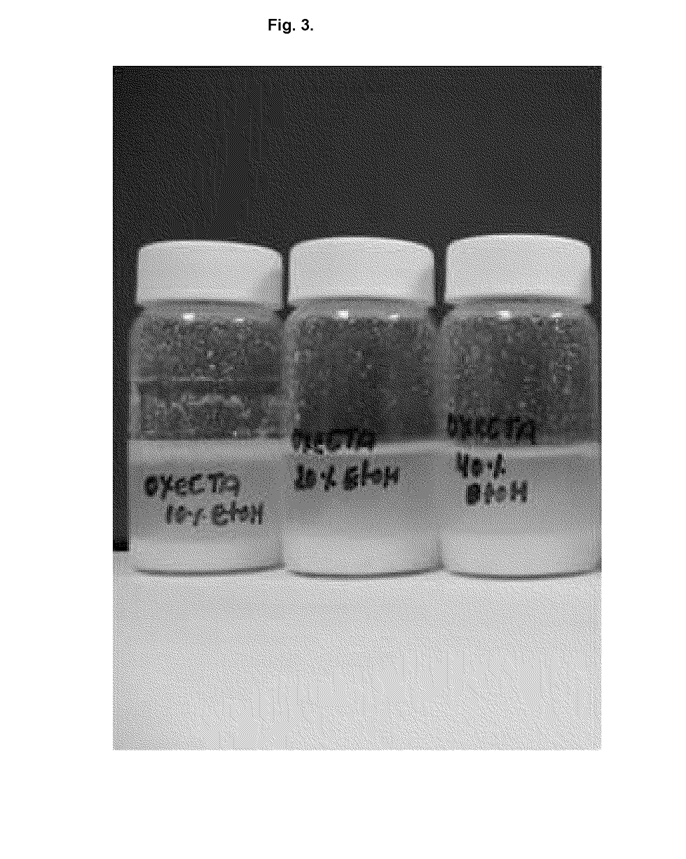

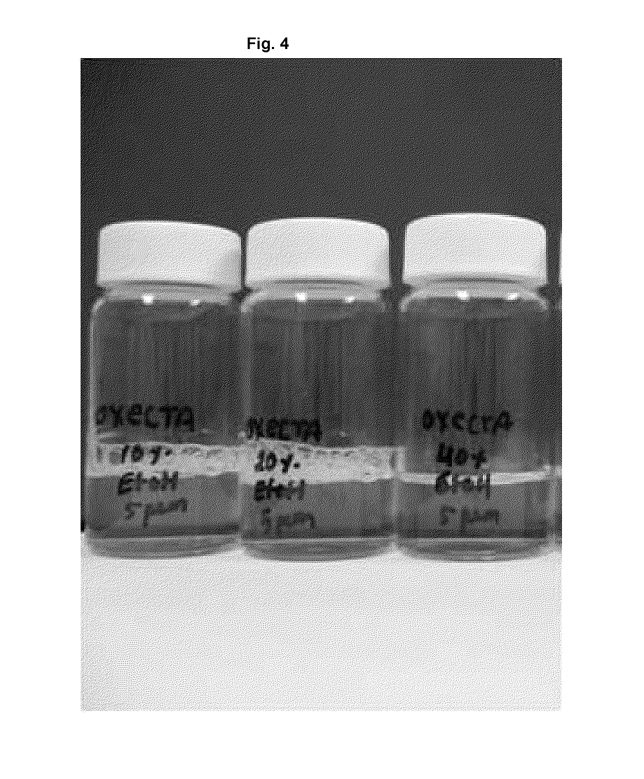

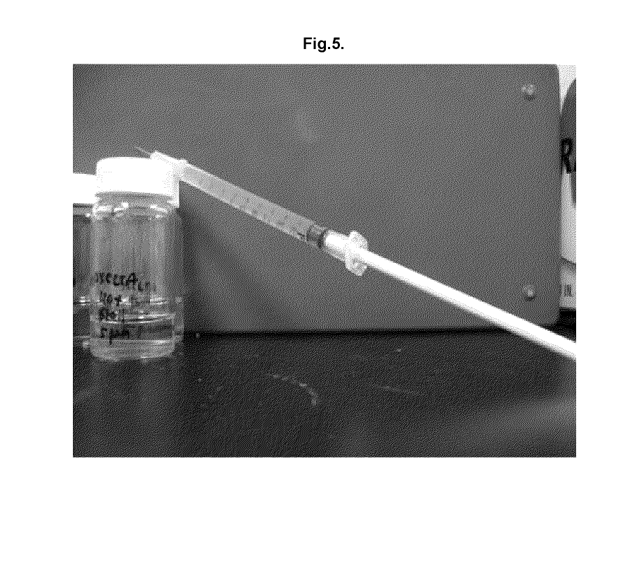

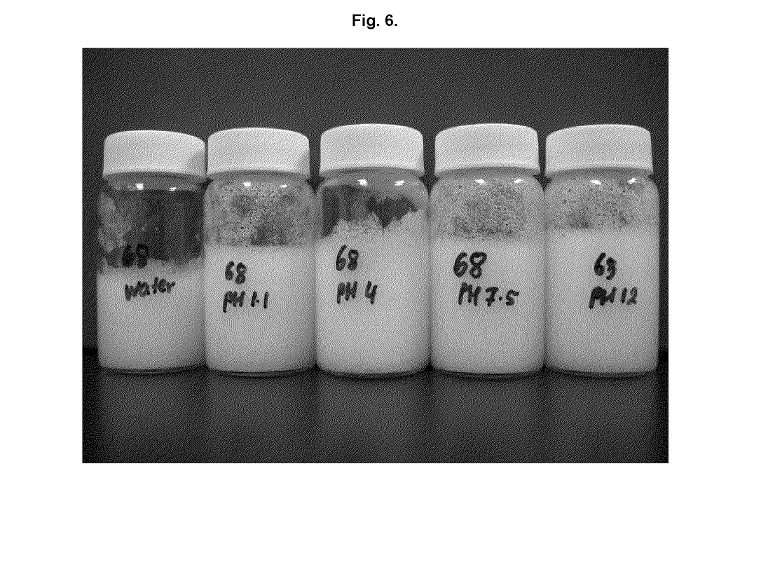

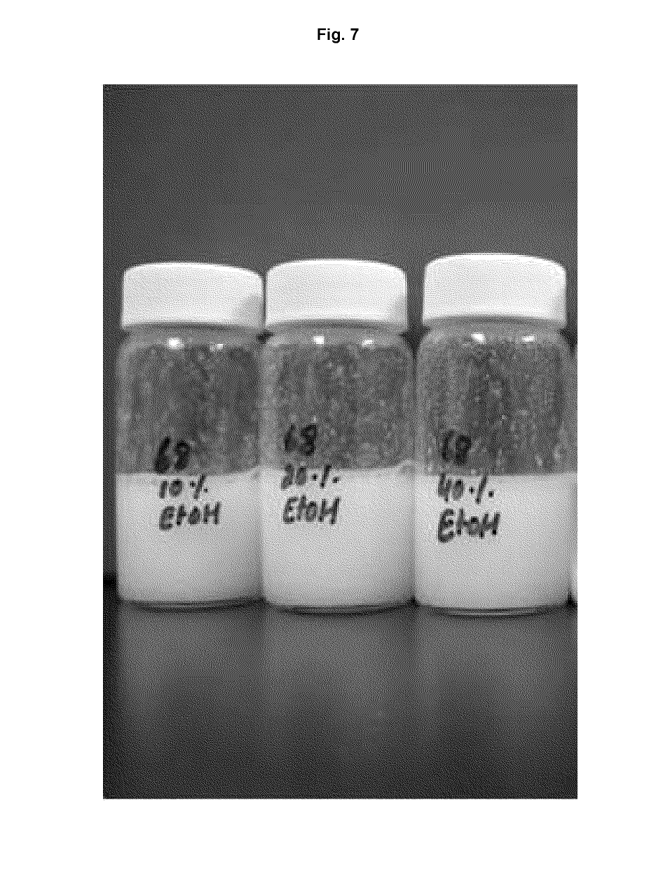

In order to assess the effectiveness of the formulation of Example 2 to deter potential abusers from extracting an opioid substance (hydromorphone) from an immediate release formulation, tests were carried out to determine the syringeability, injectability, filtration and gelation time of a crushed tablet from Example 2 in 10 ml of various media solvent light shaking 20 times at room temperature comparative to Oxecta® (an immediate release oral formulation of Oxycodone). Results are shown in FIGS. 1 to 7.

TABLE 5
Syringeability, injectability, filterability and
gelation time of crushed tablet from Example 2.

| | | Semi- | Syringeability, injectability, filtration and gelation time | |
|---|---|---|---|---|
| Media | Liquid | solid | Example 2 | Oxecta ® - IR |
| Water | ✓ | x | Initially thick viscous fluid | 2-4 min -phase separation occurs |
| | | | gel occurs which after 3-5 | with a liquid upper layer and a |
| | | | minutes turns into solid | solid cake of insoluble ingredients |
| | | | | at the bottom/The top layer was |
| | | | | syringeable & injectable through |
| | | | | insulin syringe needle. |
| 0.1N | ✓ | x | It was almost solid but | 4 min -phase separation occurs |
| pH 1.1 | | | flow able in pH 1.1 | with a liquid upper layer and a |
| | | | | solid cake of insoluble ingredients |
| | | | | at the bottom./The top layer was |
| | | | | syringeable & injectable through |
| | | | | insulin syringe needle. |
| Acetate | ✓ | x | Initially thick viscous fluid | 4 min -phase separation occurs |
| Buffer | | | gel occurs which after 3-5 | with a liquid upper layer and a |
| pH 4 | | | minutes turns into solid | solid cake of insoluble ingredients |
| | | | | at the bottom/The top layer was |
| | | | | syringeable & injectable through |
| | | | | insulin syringe needle. |
| Phosphate | ✓ | x | Initially thick viscous fluid | 4 min -phase separation occurs |
| buffer | | | gel occurs which after 3-5 | with a liquid upper layer and a |
| pH 7.5 | | | minutes turns into solid | solid cake of insoluble ingredients |
| | | | | at the bottom./The top layer was |
| | | | | syringeable & injectable through |
| | | | | insulin syringe needle. |
| 0.5% w/v | ✓ | x | Initially thick viscous fluid | the top layer was syringeable & |
| NaOH | | | gel occurs which after 3-5 | injectable through insulin syringe |
| pH 12.0 | | | minutes turns into solid | needle. |
| 40% v/v | ✓ | x | The uniform mixture at | Filtered top layer in 40% v/v |
| ethanol | | | 40% v/v Ethanol or below | ethanol was syringeable and |
| | | | was not syringeable, | injectable |
| | | | injectable and filtrable. | |

Observation:

(✓/x indicated the yes/no for the respective physical form of the mixture of crushed powder and the media immediately after the addition of the media at time zero). It was filterable through a 5 micron syringe filter

### Solvent Light Shaking 20 Times

After 5 minutes no phase separation occurs in 10%, 20%, 40% v/v ethanol and above, with a liquid upper layer and a solid cake of insoluble ingredients at the bottom in sample of Example 2;
After 2 minutes phase separation occurs with a liquid upper layer and a solid cake of insoluble ingredients at the bottom in samples of Oxecta®.

### Syringeability, Injectability and Filtration

Initially thick viscous fluid gel which after 3-5 minutes turns into solid in water, pH 4, pH 7.5 & pH 12. It was almost solid but flowable in pH 1.1. The uniform mixture at 10%, 20%, 40% v/v Ethanol below was not syringeable, injectable and filtrable. When this was loaded from the back of the plunger and forced through insulin syringe or 21 G big needle, the lock failed resulting in gel spillover. It cannot pass through such needles even with high applied force,
In solvents, the top layer was syringeable & injectable through insulin syringe needle. It was filterable through 5 micron syringe filter. Filtered top layer in 40% v/v ethanol was syringeable and injectable in samples of Oxecta®.

### Solvent Light Shaking 20 Times

Oxecta: after 2 to 4 minutes phase separation occurs with a liquid upper layer and a solid cake of insoluble ingredients at the bottom in samples of Oxecta® in all solvents: (water; acidic, basic and 40% v/v ethanol).

### Example 2

initially thick viscous fluid gel occurs, which after 3-5 minutes turns into solid mass in all solvents (water, acidic, basic and ethanol). The uniform viscous mixture occurs at 40% v/v.

### Syringability, Injectability and Filterability

Oxecta®:

after 2 to 4 minutes phase separation occurs with a liquid upper layer and a solid cake of insoluble ingredients at the bottom in samples of Oxecta® in all solvents. It was filterable through 5 micron syringe filter. The top layer was syringable & injectable through insulin syringe needle. Filtered top layer in 40% v/v ethanol was syringable and injectable in samples of Oxecta®.

### Example 2

Initially thick viscous fluid gel which after 3-5 minutes turns into solid in water, pH 4, pH 7.5 & pH 12. It was almost solid but flow able in pH 1.1. It was non-filterable through 5 micron syringe filter, non-syringeable and non-injectable. The uniform viscous mixture at 40% v/v Ethanol or below was not syringeable, injectable and filtrable. When this was loaded from the back of the plunger and forced through insulin syringe or 21G big needle, the lock failed with the gel spillover. It cannot pass through such needles even with high applied force, in sample of Example 7.

### Example 3

### Tablet Preparation

The procedure of Example 1 is reproduced in this example with Zolpidem as API. In the present example, xanthan gum and konjac glucomannan are used. A surfactant is also used.

The formulation of Example 3 is set out in Table 6.

TABLE 6
Formulation of Example 3.

Qty/Tab

| N | Ingredient | (mg) | % w/w |
|---|---|---|---|
| 1 | Zolpidem | 5 | 1.25 |
| 2 | Sodium lauryl sulfate | 20.0 | 5.0 |
| 3 | Xanthan gum 180 | 20.0 | 5.0 |
| 4 | Konjac glucomannan | 50.0 | 12.5 |
| 5 | Crospovidone XL | 40.0 | 10.0 |
| 6 | Magnesium stearate | 4.0 | 1.0 |
| 7 | Microcrystalline | 261.0 | 65.25 |
| | cellulose pH 102 | | |
| | Total Core | 400 | 100 |

The tablets is monitored for weight, hardness, thickness and friability. The tablets are tested for assay, release characteristics (in vitro dissolution method) and the abuse deterrent properties.

### Example 4

### Tablet Preparation

The procedure of Example 1 was reproduced in this example with Hydromorphone HCl as API. In the present example, xanthan gum and konjac glucomannan are used. A surfactant is also used.

The formulation of Example 4 is set out in Table 7.

TABLE 7
Formulation an abuse-deterrent immediate-
release Oxycodone HCl - Example 4.

Qty/Tab

| N | Ingredient | (mg) | % w/w |
|---|---|---|---|
| 1 | Oxycodone HCl | 7.5 | 1.87 |
| 2 | Sodium lauryl sulfate | 20.0 | 5.0 |
| 3 | Xanthan gum 180 | 20.0 | 5.0 |
| 4 | Konjac glucomannan | 50.0 | 12.5 |
| 5 | Crospovidone XL | 40.0 | 10.0 |
| 6 | Magnesium stearate | 4.0 | 1.0 |
| 7 | Microcrystalline | 258.5 | 64.6 |
| | cellulose pH 102 | | |
| | Total Core | 400 | 100 |

The tablets are monitored for weight, hardness, thickness and friability. The tablets are tested for assay, release characteristics (in vitro dissolution method) and abuse deterrent properties.

### Example 5

### Tablet Preparation

The procedure of Example 1 is reproduced in this example with Oxycodone HCl as API. In the present example, gellan gum and konjac glucomannan are used. A surfactant is also used.

The formulation of Example 5 is set out in Table 8.

TABLE 8
Abuse deterrent formulation of Example 5.

Qty/Tab

| N | Ingredient | (mg) | % w/w |
|---|---|---|---|
| 1 | Oxycodone HCl | 7.93 | 1.98 |
| 2 | Sodium lauryl sulfate | 7.0 | 1.75 |
| 3 | Gellan gum CG-HA | 5.0 | 1.25 |
| 4 | Konjac glucomannan | 5.0 | 1.25 |
| 5 | HPMC E10 | 5.0 | 1.25 |
| 6 | Crospovidone XL | 40.0 | 10.0 |
| 7 | Magnesium stearate | 4.0 | 1.0 |
| 8 | Microcrystalline | 326.1 | 81.51 |
| | cellulose pH 102 | | |
| | Total Core | 400 | 100 |

The tablets are monitored for weight, hardness, thickness and friability. The tablets are tested for assay, release characteristics (in vitro dissolution method) and abuse deterrent properties.

### Example 6

### Tablet Preparation

The procedure of Example 1 was reproduced example Hydromorphone HCl as API deterrent. In the present example, xanthan gum and konjac glucomannan are used. A surfactant was also used.

The formulation Example 6 is set out in Table 9.

TABLE 9
Formulation of immediate release Hydromorphone of Example 6.

Qty/Tab

| N | Ingredient | (mg) | % w/w |
|---|---|---|---|
| 1 | Hydromorphone HCl | 8.0 | 2.0 |
| 2 | Sodium lauryl sulfate | 20.0 | 5.0 |
| 3 | Xanthan gum 180 | 20.0 | 5.0 |
| 4 | Konjac glucomannan | 50.0 | 12.5 |
| 5 | Crospovidone XL | 40.0 | 10.0 |
| 6 | Magnesium stearate | 4.0 | 1.0 |
| 7 | Microcrystalline | 258.0 | 64.5 |
| | cellulose pH 102 | | |
| | Total Core | 400 | 100 |

The tablets were monitored for weight, hardness, thickness and friability. The tablets were tested for assay, release characteristics (in vitro dissolution method) and abuse deterrent properties.

### Evaluation of Gelation Behaviour

Each coated tablet was crushed in a mortar and pestle to get fine powder. The resulting powder was then transferred to 20 mL clear glass vial and 10 mL of solution media was added. It was stirred immediately vigorously and the time taken to get a mass that did not fall while inverting the bottle was noted.

### Example 7

### Tablet Preparation

The procedure of Example 1 was reproduced in this example with hydromorphone HCl as API. In the present example the formulation comprises: xanthan gum and a konjac glucomannan. Xanthan gum was selected as pH independent gum, konjac glucomannan gels in all solvents. Also, is used a surfactant. The formulation Example 7 is set out in Table 10.

TABLE 10
Formulation of immediate release Hydromorphone of Example 7.

Qty/Tab

| N | Ingredient | (mg) | % w/w |
|---|---|---|---|
| 1 | Hydromorphone HCl | 8.0 | 1.6 |
| 2 | Sodium lauryl sulfate | 20.0 | 4.0 |
| 3 | Xanthan gum 180 | 25.0 | 5.0 |
| 4 | Konjac glucomannan | 70.0 | 14.0 |
| 5 | Crospovidone XL | 50.0 | 10.0 |
| 6 | Magnesium stearate | 5.0 | 1.0 |
| 7 | Microcrystalline | 322.0 | 64.4 |
| | cellulose pH 102 | | |
| | Total Core | 500 | 100 |

The tablets were monitored for weight, hardness, thickness and friability. The tablets were tested for assay, release characteristics (in vitro dissolution method) and abuse deterrent properties.

### Evaluation of Dissolution Profile

The pharmaceutical dosage form obtained from Example 7 was subsequently tested for in vitro dissolution rate, measured by Apparatus (USP Type II with paddle), using the following parameters:
Media: 500 ml of purified water
Speed: 50 rpm
Temperature: at 37 deg. C

The acceptable dissolution criterion is not less than 75% of the drug dissolved in 45 minutes. (U.S. Pharmacopoeia, XXVI, 2003).

### Evaluation of Gelation Behaviour

Each coated tablet was crushed in a mortar and pestle to get a fine powder. This was then transferred to a 20 mL clear glass vial and 10 mL of solution media was added. It was stirred immediately vigorously and the time taken to get a mass that did not fall while inverting the bottle was noted.

### Syringeability and Injectability

In order to abuse the drugs via injection route, abusers typically crush the tablet and dissolve in small amount of water to extract the soluble drug. The ease in the drawing of the mass into the syringe (syringeability) and injection of the mass in the syringe (injectability) was determined using the insulin syringe which they typically use. Crushed tablets of Example 7 quickly turned into a solid gel-like mass within a few minutes in the cold media and within a minute in the hot media that did not fall upon inversion of the vial within few minutes in cold media and within a minute in hot media. Due to such solid-like consistency of crushed tablets in those media (Table 11), it was not possible to draw the mass into a syringe for subsequent injection.

In order to assess the effectiveness of present formulation to deter potential abusers from extracting an opioid substance from an immediate release formulation, gelation tests were carried out to determine the time to gelation of a crushed tablet from Example 7 in 10 ml of media at room temperature (Table 11). Time taken to get a mass that did not fall while inverting the glass vial at 180° was noted. The faster this solid mass is formed, the lower are the chances of drawing the solution and injecting by potential abusers is. It was noted that the gelation time was within 3 minutes in non-alcoholic media covering almost the whole pH ranges. Since abusers typically try to dissolve the drug in water, the quick gelation time in water was of added value. Similarly, the tablet formula gelled at 10% ethanol in water. In higher ethanol concentration, a thick liquid viscous fluid mass was obtained. However, in those cases, the viscous fluid mass fell while inverting the glass vial to 180°.

In order to assess the effectiveness of the formulation of Example 7 to deter potential abusers from extracting an opioid substance (hydromorphone) from an immediate release formulation, tests were carried out to determine the syringeability, injectability, filtration and gelation time of a crushed tablet from Example 7 in 10 ml of various media solvent light shaking 20 times at room temperature comparative to Oxecta® (an immediate release oral formulation of Oxycodone). Results are shown in FIGS. 1 to 7.

TABLE 11
Solubility, filtration, syringeability, injectability,
and gelation time of crushed tablet from Example 7.

| | | Semi- | Syringeability, injectability, filtration and gelation time | |
|---|---|---|---|---|
| Media | Liquid | solid | Example 7 | Oxecta ® - IR |
| Water | ✓ | x | Initially thick viscous fluid | 2-4 min -phase separation occurs |
| | | | gel occurs which after 3-5 | with a liquid upper layer and a |
| | | | minutes turns into solid | solid cake of insoluble ingredients |
| | | | | at the bottom/The top layer was |
| | | | | syringeable & injectable through |
| | | | | insulin syringe needle. |
| 0.1N | ✓ | x | It was almost solid but | 4 min -phase separation occurs |
| pH 1.1 | | | flow able in pH 1.1 | with a liquid upper layer and a |
| | | | | solid cake of insoluble ingredients |
| | | | | at the bottom./The top layer was |
| | | | | syringeable & injectable through |
| | | | | insulin syringe needle. |
| Acetate | ✓ | x | Initially thick viscous fluid | 4 min -phase separation occurs |
| Buffer | | | gel occurs which after 3-5 | with a liquid upper layer and a |
| pH 4 | | | minutes turns into solid | solid cake of insoluble ingredients |
| | | | | at the bottom/The top layer was |
| | | | | syringeable & injectable through |
| | | | | insulin syringe needle. |
| Phosphate | ✓ | x | Initially thick viscous fluid | 4 min -phase separation occurs |
| buffer | | | gel occurs which after 3-5 | with a liquid upper layer and a |
| pH 7.5 | | | minutes turns into solid | solid cake of insoluble ingredients |
| | | | | at the bottom./The top layer was |
| | | | | syringeable & injectable through |
| | | | | insulin syringe needle. |
| 0.5% w/v | ✓ | x | Initially thick viscous fluid | the top layer was syringeable & |
| NaOH | | | gel occurs which after 3-5 | injectable through insulin syringe |
| pH 12.0 | | | minutes turns into solid | needle. |
| 40% v/v | ✓ | x | The uniform mixture at | Filtered top layer in 40% v/v |
| ethanol | | | 40% v/v Ethanol or below | ethanol was syringeable and |
| | | | was not syringeable, | injectable |
| | | | injectable and filtrable. | |

Observation:

(✓/x indicated the yes/no for the respective physical form of the mixture of crushed powder and the media immediately after the addition of the media at time zero). It was filterable through a 5 micron syringe filter

### Solvent Light Shaking 20 Times

After 5 minutes no phase separation occurs in 10%, 20%, 40% v/v ethanol and above, with a liquid upper layer and a solid cake of insoluble ingredients at the bottom in sample of Example 7;
After 2 minutes phase separation occurs with a liquid upper layer and a solid cake of insoluble ingredients at the bottom in samples of Oxecta®.

### Syringeability, Injectability and Filterability

Initially thick viscous fluid gel which after 3-5 minutes turns into solid in water, pH 4, pH 7.5 & pH 12. It was almost solid but flow able in pH 1.1. The uniform mixture at 10%, 20%, 40% v/v Ethanol below was not syringeable, injectable and filtrable. When this was loaded from the back of the plunger and forced through insulin syringe or 21G big needle, the lock failed resulting in gel spillover. It cannot pass through such needles even with high applied force,
In solvents, the top layer was syringeable & injectable through insulin syringe needle. It was filterable through 5 micron syringe filter. Filtered top layer in 40% v/v ethanol was syringeable and injectable in samples of Oxecta®.

### Solvent Light Shaking 20 Times

Oxecta:

after 2 to 4 minutes phase separation occurs with a liquid upper layer and a solid cake of insoluble ingredients at the bottom in samples of Oxecta® in all solvents: (water; acidic, basic and 40% v/v ethanol).

### Example 7

initially thick viscous fluid gel occurs, which after 3-5 minutes turns into solid mass in all solvents (water, acidic, basic and ethanol). The uniform viscous mixture occurs at 40% v/v.

### Syringability, Injectability and Filterability

Oxecta:

after 2 to 4 minutes phase separation occurs with a liquid upper layer and a solid cake of insoluble ingredients at the bottom in samples of Oxecta® in all solvents. It was filterable through 5 micron syringe filter. The top layer was syringable & injectable through insulin syringe needle. Filtered top layer in 40% v/v ethanol was syringable and injectable in samples of Oxecta®.

### Example 7

Initially thick viscous fluid gel which after 3-5 minutes turns into solid in water, pH 4, pH 7.5 & pH 12. It was almost solid but flow able in pH 1.1. It was non-filterable through 5 micron syringe filter, non-syringeable and non-injectable. The uniform viscous mixture at 40% v/v Ethanol or below was not syringeable, injectable and filtrable. When this was loaded from the back of the plunger and forced through insulin syringe or 21 G big needle, the lock failed with the gel spillover. It cannot pass through such needles even with high applied force, in sample of Example 7.

### Example 8

### Tablet Preparation

The procedure of Example 1 was reproduced in this example with Hydromorphone HCl as API. In the present example the formulation comprises: xanthan gum, konjac glucomannan and gellan gum. A surfactant was also used.

The formulation Example 8 is set out in Table 12.

TABLE 12
Formulation of immediate release Hydromorphone of Example 8.

Qty/Tab

| N | Ingredient | (mg) | % w/w |
|---|---|---|---|
| 1 | Hydromorphone HCl | 8.0 | 1.6 |
| 2 | Sodium lauryl sulfate | 20.0 | 4.0 |
| 3 | Xanthan gum 180 | 10.0 | 2.0 |
| 4 | Konjac glucomannan | 70.0 | 14.0 |
| 5 | Gellan gum | 15.0 | 3.0 |
| 6 | Crospovidone XL | 50.0 | 10.0 |
| 7 | Magnesium stearate | 5.0 | 1.0 |
| 8 | Microcrystalline | 322.0 | 64.4 |
| | cellulose pH 102 | | |
| | Total Core | 500 | 100 |

The tablets were monitored for weight, hardness, thickness and friability. The tablets were tested for assay, release characteristics (in vitro dissolution method) and abuse deterrent properties.

### Evaluation of Gelation Behaviour

Each coated tablet was crushed in a mortar and pestle to get fine powder. This is then transferred to 20 mL clear glass vial and 10 mL of solution media was added. It was stirred immediately vigorously and the time taken to get a mass that did not fall while inverting the bottle was noted.

### Example 9

### Tablet Preparation

To make an abuse-deterrent immediate release formulation of Oxycodone HCl the following manufacturing steps were followed:

Step 1:

The required quantity of Oxycodone HCl (7.9 mg) was mixed with required quantity of konjac glucomannan (10.0 mg). Was added to the mixed blend the required quantity of gellan gum (10.0 mg) and required quantity of HPMC (10.0 mg) and also was mixed thoroughly. Then, the required quantity of sodium lauryl sulfate (7.0 mg) and the required quantity of crospovidone XL (40.0 mg), were added and were mixed thoroughly.

Step 2:

Blend obtained from previous step was mixed with % of the required quantity of microcrystalline cellulose (311.0 mg). The remaining ½ of the required quantity of microcrystalline cellulose was added and was mixed thoroughly. Then, the blend obtained was passed through a 40 mesh sieve.

Step 3:

The required quantity of magnesium stearate (4.0 mg) was mixed with 50 grams of blend from step 2 and passed through a 40 mesh sieve. The remaining mixture of step 2 was added and mixed for 30 seconds to 1 minute. Then, the blend obtained was direct compressed.

The formulation of Example 9 is set out in Table 13.

TABLE 13
Formulation of the abuse deterrent immediate
release Oxycodone of Example 9.

Qty/Tab

| N | Ingredient | (mg) | % w/w |
|---|---|---|---|
| 1 | Oxycodone HCl | 7.93 | 1.98 |
| 2 | Sodium lauryl sulfate | 7.0 | 1.75 |
| 3 | Gellan gum CG-HA | 10.0 | 2.5 |
| 4 | Konjac glucomannan | 10.0 | 2.5 |
| 5 | HPMC E10 | 10.0 | 2.5 |
| 6 | Crospovidone XL | 40.0 | 10.0 |
| 7 | Magnesium stearate | 4.0 | 1.0 |
| 8 | Microcrystalline | 311.1 | 77.76 |
| | cellulose pH 102 | | |
| | Total Core | 400 | 100 |

The tablets were monitored for weight, hardness, thickness and friability. The tablets were tested for assay, release characteristics (in vitro dissolution method) and abuse deterrent properties.

### Evaluation of Dissolution Profile

The pharmaceutical dosage form obtained from Example 9 was subsequently tested for in vitro dissolution rate, measured by Apparatus (USP Type II with paddles), using the following parameters:
Media: 500 ml of purified water
Speed: 50 rpm
Temperature: at 37 deg. C

The acceptable dissolution criterion is not less than 75% of the drug dissolved in 45 minutes. (U.S. Pharmacopoeia, XXVI, 2003)

### Example 10

### Tablet Preparation

The procedure of Example 1 is reproduced in this example with Amphetamine as API.

The formulation of Example 10 is set out in Table 14.

TABLE 14
Formulation of Example 10.

Qty/Tab

| N | Ingredient | (mg) | % w/w |
|---|---|---|---|
| 1 | Amphetamine | 10.0 | 2.5 |
| 2 | Sodium lauryl sulfate | 20.0 | 5.0 |
| 3 | Xanthan gum 180 | 20.0 | 5.0 |
| 4 | Konjac glucomannan | 50.0 | 12.5 |
| 5 | Crospovidone XL | 40.0 | 10.0 |
| 6 | Magnesium stearate | 4.0 | 1.0 |
| 7 | Microcrystalline | 256.0 | 64.0 |
| | cellulose pH 102 | | |
| | Total Core | 400 | 100 |

The tablets is monitored for weight, hardness, thickness and friability. The tablets is tested for assay, release characteristics (in vitro dissolution method) and abuse deterrent properties.

### Example 11

### Tablet Preparation

The procedure of Example 1 is reproduced in this example with Methylphenidate as API.

The formulation of Example 11 is set out in Table 15.

TABLE 15
Formulation of Example 11.

Qty/Tab

| N | Ingredient | (mg) | % w/w |
|---|---|---|---|
| 1 | Methylphenidate | 10.0 | 2.5 |
| 2 | Sodium lauryl sulfate | 20.0 | 5.0 |
| 3 | Xanthan gum 180 | 20.0 | 5.0 |
| 4 | Konjac glucomannan | 50.0 | 12.5 |
| 5 | Crospovidone XL | 40.0 | 10.0 |
| 6 | Magnesium stearate | 4.0 | 1.0 |
| 7 | Microcrystalline | 256.0 | 64.0 |
| | cellulose pH 102 | | |
| | Total Core | 400 | 100 |

The tablets are monitored for weight, hardness, thickness and friability. The tablets are tested for assay, release characteristics (in vitro dissolution method) and abuse deterrent properties.

### Example 12

### Tablet Preparation

The procedure of Example 1 is reproduced in this example with morphine as API.

The formulation of Example 12 is set out in Table 16.

TABLE 16
Formulation of Example 12.

Qty/Tab

| N | Ingredient | (mg) | % w/w |
|---|---|---|---|
| 1 | Morphine HCl | 30.0 | 7.5 |
| 2 | Sodium lauryl sulfate | 20.0 | 5.0 |
| 3 | Xanthan gum 180 | 20.0 | 5.0 |
| 4 | Konjac glucomannan | 50.0 | 12.5 |
| 5 | Crospovidone XL | 40.0 | 10.0 |
| 6 | Magnesium stearate | 4.0 | 1.0 |
| 7 | Microcrystalline | 236.0 | 59.0 |
| | cellulose pH 102 | | |
| | Total Core | 400 | 100 |

The tablets are monitored for weight, hardness, thickness and friability. The tablets are tested for assay, release characteristics (in vitro dissolution method) and abuse deterrent properties.

### Example 13

### Tablet Preparation

The procedure of Example 1 is reproduced in this example with fentanyl as API.

The formulation of Example 13 is set out in Table 17.

TABLE 17
Formulation of Example 13.

Qty/Tab

| N | Ingredient | (mg) | % w/w |
|---|---|---|---|
| 1 | Fentanyl | 0.2 | 0.05 |
| 2 | Sodium lauryl sulfate | 20.0 | 5.0 |
| 3 | Xanthan gum 180 | 20.0 | 5.0 |
| 4 | Konjac glucomannan | 50.0 | 12.5 |
| 5 | Crospovidone XL | 40.0 | 10.0 |
| 6 | Magnesium Stearate | 4.0 | 1.0 |
| 7 | Microcrystalline | 265.8 | 66.45 |
| | Cellulose pH 102 | | |
| | Total Core | 400 | 100 |

The tablets are monitored for weight, hardness, thickness and friability. The tablets are tested for assay, release characteristics (in vitro dissolution method) and the abuse deterrent properties.

### Example 14

### Tablet Preparation

The procedure of Example 1 is reproduced in this example with Hydrocodone as API.

The formulation of Example 14 is set out in Table 18.

TABLE 18
Formulation of Example 14.

Qty/Tab

| N | Ingredient | (mg) | % w/w |
|---|---|---|---|
| 1 | Hydrocodone | 10.0 | 2.5 |
| 2 | Sodium lauryl sulfate | 20.0 | 5.0 |
| 3 | Xanthan gum 180 | 20.0 | 5.0 |
| 4 | Konjac glucomannan | 50.0 | 12.5 |
| 5 | Crospovidone XL | 40.0 | 10.0 |
| 6 | Magnesium stearate | 4.0 | 1.0 |
| 7 | Microcrystalline | 256.0 | 64.0 |
| | cellulose pH 102 | | |
| | Total Core | 400 | 100 |

The tablets are monitored for weight, hardness, thickness and friability. The tablets are tested for assay, release characteristics (in vitro dissolution method) and the abuse deterrent properties.

### Example 15

### Tablet Preparation

The procedure of Example 1 is reproduced in this example with alprazolam as API.

The formulation of Example 15 is set out in Table 19.

TABLE 19
Formulation of Example 15.

| | | Qty/Tab | | Qty/Batch (g) | |
|---|---|---|---|---|---|
| N | Ingredient | (mg) | % w/w | Theor. | Weighed |
| 1 | Alprazolam | 0.5 | 0.125 | 0.25 | |
| 2 | Sodium lauryl sulfate | 20.0 | 5.0 | 10.0 | |
| 3 | Xanthan gum 180 | 20.0 | 5.0 | 10.0 | |
| 4 | Konjac glucomannan | 50.0 | 12.5 | 25.0 | |
| 5 | Crospovidone XL | 40.0 | 10.0 | 20.0 | |
| 6 | Magnesium stearate | 4.0 | 1.0 | 2.0 | |
| 7 | Microcrystalline | 265.5 | 66.37 | 132.75 | |
| | cellulose pH 102 | | | | |
| | Total Core | 400 | 100 | 200 | |

The tablets are monitored for weight, hardness, thickness and friability. The tablets are tested for assay, release characteristics (in vitro dissolution method) and the abuse deterrent properties.

### Example 16

### Tablet Preparation

The procedure of Example 1 is reproduced in this example with Diazepam as API

The formulation of Example 16 is set out in Table 20.

TABLE 20
Formulation of Example 16.

Qty/Tab

| N | Ingredient | (mg) | % w/w |
|---|---|---|---|
| 1 | Diazepam | 5 | 1.25 |
| 2 | Sodium lauryl sulfate | 20.0 | 5.0 |
| 3 | Xanthan gum 180 | 20.0 | 5.0 |
| 4 | Konjac glucomannan | 50.0 | 12.5 |
| 5 | Crospovidone XL | 40.0 | 10.0 |
| 6 | Magnesium stearate | 4.0 | 1.0 |
| 7 | Microcrystalline | 261.0 | 65.25 |
| | cellulose pH 102 | | |
| | Total Core | 400 | 100 |

The tablets are monitored for weight, hardness, thickness and friability. The tablets are tested for assay, release characteristics (in vitro dissolution method) and the abuse deterrent properties.

### Example 17

### Tablet Preparation

The procedure of Example 1 was reproduced in this example with Zopiclone as API.

The formulation of Example 17 is set out in Table 21.

TABLE 21
Formulation of Example 17.

Qty/Tab

| N | Ingredient | (mg) | % w/w |
|---|---|---|---|
| 1 | Zopiclone | 7.5 | 1.87 |
| 2 | Sodium lauryl sulfate | 20.0 | 5.0 |
| 3 | Xanthan gum 180 | 20.0 | 5.0 |
| 4 | Konjac glucomannan | 50.0 | 12.5 |
| 5 | Crospovidone XL | 40.0 | 10.0 |
| 6 | Magnesium stearate | 4.0 | 1.0 |
| 7 | Microcrystalline | 258.5 | 64.62 |
| | cellulose pH 102 | | |
| | Total Core | 400 | 100 |

The tablets were monitored for weight, hardness, thickness and friability. The tablets were tested for assay, release characteristics (in vitro dissolution method) and the abuse deterrent properties.

### Example 18

### Tablet Preparation

The procedure of Example 1 is reproduced in this example with Temazepam as API.

The formulation of Example 18 is set out in Table 22.

TABLE 22
Formulation of Example 18.

Qty/Tab

| N | Ingredient | (mg) | % w/w |
|---|---|---|---|
| 1 | Temazepam | 15.0 | 3.75 |
| 2 | Sodium lauryl sulfate | 20.0 | 5.0 |
| 3 | Xanthan gum 180 | 20.0 | 5.0 |
| 4 | Konjac glucomannan | 50.0 | 12.5 |
| 5 | Crospovidone XL | 40.0 | 10.0 |
| 6 | Magnesium stearate | 4.0 | 1.0 |
| 7 | Microcrystalline | 251.0 | 62.75 |
| | cellulose pH 102 | | |
| | Total Core | 400 | 100 |

The tablets are monitored for weight, hardness, thickness and friability. The tablets are tested for assay, release characteristics (in vitro dissolution method) and the abuse deterrent properties

### Example 19

### Tablet Preparation

The procedure of Example 1 is reproduced in this example with Nitrazepam as API.

The formulation of Example 19 is set out in Table 23.

TABLE 23
Formulation of Example 19.

Qty/Tab

| N | Ingredient | (mg) | % w/w |
|---|---|---|---|
| 1 | Nitrazepam | 10.0 | 2.5 |
| 2 | Sodium lauryl sulfate | 20.0 | 5.0 |
| 3 | Xanthan gum 180 | 20.0 | 5.0 |
| 4 | Konjac glucomannan | 50.0 | 12.5 |
| 5 | Crospovidone XL | 40.0 | 10.0 |
| 6 | Magnesium stearate | 4.0 | 1.0 |
| 7 | Microcrystalline | 256.0 | 64.0 |
| | cellulose pH 102 | | |
| | Total Core | 400 | 100 |

The tablets are monitored for weight, hardness, thickness and friability. The tablets are tested for assay, release characteristics (in vitro dissolution method) and the abuse deterrent properties.

### Example 20

### Tablet Preparation

The procedure of Example 1 is reproduced in this example with Lorazepam as API.

The formulation of Example 20 is set out in Table 24.

TABLE 24
Formulation of Example 20.

| | | Qty/Tab | | Qty/Batch (g) | |
|---|---|---|---|---|---|
| N | Ingredient | (mg) | % w/w | Theor. | Weighed |
| 1 | Lorazepam | 1.0 | 0.25 | 0.5 | |
| 2 | Sodium lauryl sulfate | 20.0 | 5.0 | 10.0 | |
| 3 | Xanthan gum 180 | 20.0 | 5.0 | 10.0 | |
| 4 | Konjac glucomannan | 50.0 | 12.5 | 25.0 | |
| 5 | Crospovidone XL | 40.0 | 10.0 | 20.0 | |
| 6 | Magnesium stearate | 4.0 | 1.0 | 2.0 | |
| 7 | Microcrystalline | 265.0 | 66.25 | 132.5 | |
| | cellulose pH 102 | | | | |
| | Total Core | 400 | 100 | 200 | |

The tablets are monitored for weight, hardness, thickness and friability. The tablets are tested for assay, release characteristics (in vitro dissolution method) and the abuse deterrent properties.

1. An immediate release orally administrable abuse-deterrent pharmaceutical formulation comprising:
a) at least one pharmaceutically active ingredient susceptible to abuse within a matrix;
b) at least one gelling polymeric compound, selected from a group consisting of: a natural resin, a natural gum, a polymer and a combination thereof;
c) at least one disintegrant; and
d) optionally a surfactant.

2. (canceled)

3. An immediate release orally administrable abuse deterrent pharmaceutical formulation comprising: at least one pharmaceutically active ingredient susceptible to abuse; konjac glucomanan; at least one other gelling polymeric compound selected from the group consisting of: polysaccharides, sugars, sugar derived alcohols, starches, starch derivatives, cellulose derivatives, Carrageenan, pectin, sodium alginate, gellan gum, xanthan gum, poloxamer, Carbopol®, PolyOx®, povidone, hydroxypropyl methylcellulose (HPMC), hypermellose, and combinations thereof, and at least one disintegrant, wherein said formulation provides an immediate release of the pharmacologically active ingredient when the tablet is taken orally and, upon tampering, becomes an uninjectable and unsyringeable gel when exposed to aqueous, alcoholic, acidic or basic media.

4. The immediate release orally administrable abuse-deterrent pharmaceutical formulation of claim 1 further comprising at least one other pharmaceutically acceptable excipient,
wherein said formulation provides release of the active pharmaceutical ingredient and has an in vitro dissolution profile that is equal to or greater than 80 percent of the drug dissolved in 30 minutes after administration as measured by USP type I or II dissolution apparatus as described in USP XXVI (2003).

5. The immediate release orally administrable abuse-deterrent pharmaceutical formulation according to claim 1, wherein the active pharmaceutical ingredient susceptible to abuse is selected from the group consisting of: opioids and morphine derivatives; antidepressants; stimulants; hallucinogenics; hypnotics; and tranquilizers.

6. The immediate release orally administrable abuse-deterrent pharmaceutical formulation according to claim 1, wherein the active pharmaceutical ingredient susceptible to abuse is selected from the group consisting of: amphetamine, alprazolam, codeine, diazepam, fentanyl and analogs thereof, hydrocodone, hydromorphone HCl, lorazepam, meperidine, morphine, methylphenidate, methadone, nitrazepam, oxycodone HCL, oxymorphone, propoxyphene, temazepam, tramadol, zolpidem, and zopiclone.

7. The immediate release orally administrable abuse-deterrent pharmaceutical formulation according to claim 1, wherein the active pharmaceutical ingredient susceptible to abuse is present in an amount ranging from about 0.05% w/w to about 10% w/w based on the total weight of said formulation.

8. The immediate release orally administrable abuse-deterrent pharmaceutical formulation according to claim 1, wherein the active pharmaceutical ingredient susceptible to abuse is present in an amount ranging from about 0.05% w/w to about 5.0% w/w based on the total weight of said formulation.

9. The immediate release orally administrable abuse-deterrent pharmaceutical formulation according to claim 1, wherein the active pharmaceutical ingredient susceptible to abuse is present in an amount ranging from about 0.05% w/w to about 3.0% w/w based on the total weight of said formulation.

10. The immediate release orally administrable abuse-deterrent pharmaceutical formulation according to claim 1, wherein said formulation provides an in vitro dissolution profile which is equal to or greater than 75% of the active pharmaceutical ingredient susceptible to abuse dissolved in 20 minutes after administration.

11. The immediate release orally administrable abuse-deterrent pharmaceutical formulation according to claim 1, wherein said formulation provides an in vitro dissolution profile which is equal to or greater than 75% of the active pharmaceutical ingredient susceptible to abuse dissolved in 10 minutes after administration.

12. The immediate release orally administrable abuse-deterrent pharmaceutical formulation according to claim 1, wherein said formulation provides an in vitro dissolution profile which is equal to or greater than 75% of the active pharmaceutical ingredient susceptible to abuse dissolved in 30 minutes after administration.

13. The immediate release orally administrable abuse-deterrent pharmaceutical formulation according to claim 1, wherein the gelling polymeric compound is selected from the group comprising consisting of: polysaccharides, sugars, sugar derived alcohols, starches, starch derivatives, cellulose derivatives, carrageenan, pectin, sodium alginate, gellan gum, xanthan gum, poloxamer, carbopol, polyox, konjac glucomannan, povidone, hydroxypropyl methylcellulose (HPMC), hypermellose, and combinations thereof.

14. The immediate release orally administrable abuse-deterrent pharmaceutical formulation according to claim 1, wherein the gelling polymeric compound is present in an amount ranging from 1% w/w to 30% w/w.

15. (canceled)

16. The immediate release orally administrable abuse-deterrent pharmaceutical formulation according to claim 1, wherein the gelling polymeric compound is a combination of konjac glucomannan and at least one additional gelling polymeric compound and is present in an amount ranging from about 1% w/w to about 20% w/w.

17. The immediate release orally administrable abuse-deterrent pharmaceutical formulation according to claim 16, wherein the gelling polymeric compound is a combination of konjac glucomannan and an additional gelling polymeric compound selected from the group consisting of polysaccharides, sugars, sugar derived alcohols, starches, starch derivatives, cellulose derivatives, Carrageenan, pectin, sodium alginate, gellan gum, xanthan gum, poloxamer, Carbopol®, PolyOx®, povidone, hydroxypropyl methylcellulose (HPMC), hypermellose, and combinations thereof.

18. The immediate release orally administrable abuse-deterrent pharmaceutical formulation according to claim 17, wherein the ratio of konjac glucomannan to the additional gelling polymeric compound is between about 3:1 to about 9:1.

19. The immediate release orally administrable abuse-deterrent pharmaceutical formulation according to claim 1, wherein the formulation comprises a surfactant is selected from the group consisting of: sodium lauryl sulfate, poloxamer, sorbitan monoesters and glyceryl monooleates and combinations thereof.

20. The immediate release orally administrable abuse-deterrent pharmaceutical formulation according to claim 19, wherein the surfactant is sodium lauryl sulfate and is present in an amount ranging from about 1% w/w to about 10% w/w.

21. The immediate release orally administrable abuse-deterrent pharmaceutical formulation according to claim 1, wherein said formulation further comprises a nasal irritant selected from the group consisting of capsaicin, piperine, allyl isothiocyanante, sodium lauryl sulfate and combinations thereof.

22. (canceled)

23. The immediate release orally administrable abuse-deterrent pharmaceutical formulation according to claim 1, wherein said formulation comprises a therapeutically effective amount of pharmaceutically active ingredient susceptible to abuse, at least one gelling polymeric compound, at least one surfactant, along with at least one pharmaceutically acceptable excipient selected from the group consisting of: fillers, diluents, lubricants, and combinations thereof.

24. The immediate release orally administrable abuse-deterrent pharmaceutical formulation according to claim 23, wherein the filler is selected from the group consisting of: cellulose, dibasic calcium phosphate, calcium carbonate, sucrose, lactose, glucose, microcrystalline cellulose, mannitol, sorbitol, maltol, pregelatinized starch, corn starch, and combinations thereof.

25. The immediate release orally administrable abuse-deterrent pharmaceutical formulation according to claim 24, wherein the filler is microcrystalline cellulose and is present in an amount ranging from about 30% w/w to about 80% w/w based on the total weight of said formulation.

26. The immediate release orally administrable abuse-deterrent pharmaceutical formulation according to claim 1, wherein the disintegrant is selected from the group consisting of: crospovidone, sodium starch glycolate, sodium pregelatinized starch, modified corn starch and combinations thereof.

27. The immediate release orally administrable abuse-deterrent pharmaceutical formulation according to claim 26, wherein the disintegrant is crospovidone and is present in an amount ranging from about 2% w/w to about 20% w/w based on the total weight of said formulation.

28. The immediate release orally administrable abuse-deterrent pharmaceutical formulation according to claim 26, wherein the disintegrant is crospovidone and is present in an amount ranging from about 2% w/w to about 15% w/w based on the total weight of said formulation.

29. (canceled)

30. The immediate release orally administrable abuse-deterrent pharmaceutical formulation according to claim 1, wherein said formulation provides an immediate release of the active pharmaceutical ingredient susceptible to abuse when the tablet is taken orally and, upon tampering and exposure to an aqueous, alcoholic, acidic and/or basic media, said formulation becomes an uninjectable and unsyringeable gel.

31. The abuse-deterrent immediate release orally administrable pharmaceutical formulation of claim 6, wherein the formulation comprises 1% w/w to 30% w/w of the gelling polymeric compound, which comprises konjac glukomannan and at least one other gelling polymer compound selected from a natural gum, a polymer, and a combination thereof.

32. The immediate release orally administrable abuse-deterrent pharmaceutical formulation of claim 1, wherein upon exposing the formulation to aqueous, alcoholic, acidic or basic media the formulation forms a non-injectable, non-syringeable and non-filtrable gel within 5 min.

33. The immediate release orally administrable abuse-deterrent pharmaceutical formulation according to claim 16, wherein the ratio of konjac glucomannan to the additional gelling polymeric compound or combinations thereof, is between about 1:2 to about 9:2.

34. The immediate release orally administrable abuse-deterrent pharmaceutical formulation of claim 3 further comprising at least one other pharmaceutically acceptable excipient, wherein said formulation provides release of the active pharmaceutical ingredient and has an in vitro dissolution profile that is equal to or greater than 75 percent of the drug dissolved in less than 30 minutes after administration as measured by USP type I or II dissolution apparatus as described in USP XXVI (2003).

35. The immediate release orally administrable abuse-deterrent pharmaceutical formulation according to claim 34, wherein the active pharmaceutical ingredient susceptible to abuse is selected from the group consisting of: amphetamine, alprazolam, codeine, diazepam, fentanyl and analogs thereof, hydrocodone, hydromorphone HCl, lorazepam, meperidine, morphine, methylphenidate, methadone, nitrazepam, oxycodone HCL, oxymorphone, propoxyphene, temazepam, tramadol, zolpidem, and zopiclone, and is present in an amount ranging from about 0.05% w/w to about 10.0% w/w based on the total weight of said formulation.

36. The abuse-deterrent immediate release orally administrable pharmaceutical formulation according to claim 34, wherein the formulation comprises 1% w/w to 30% w/w of the gelling polymeric compound, which comprises konjac glukomannan and at least one other gelling polymer compound selected from a natural gum, a polymer, and a combination thereof.

37. The immediate release orally administrable abuse-deterrent pharmaceutical formulation according to claim 34, wherein the ratio of konjac glucomannan to the additional gelling polymeric compound or combinations thereof is between about 1:1 to about 9:1.

38. The immediate release orally administrable abuse-deterrent pharmaceutical formulation according to claim 34, wherein the ratio of konjac glucomannan to the additional gelling polymeric compound or combinations thereof is between about 1:2.

39. The immediate release orally administrable abuse-deterrent pharmaceutical formulation according to claim 34, wherein the ratio of konjac glucomannan to the additional gelling polymeric compound or combinations thereof is between about 3:1

40. The abuse-deterrent immediate release orally administrable pharmaceutical formulation of claim 3, wherein the konjac glucomannan and at least one other gelling polymer are present in an amount ranging from about 1% w/w to about 30% w/w based on the total weight of said formulation.

41. The immediate release orally administrable abuse-deterrent pharmaceutical formulation according to claim 3, wherein the ratio of konjac glucomannan to the additional gelling polymeric compound is between about 3:1 to about 9:1.

42. A method of treating a disease or disorder in a patient comprising administering to the patient an immediate release orally administrable abuse-deterrent pharmaceutical formulation of claim 1, wherein the disease or disorder is attention deficit disorder (ADD), attention deficit hyperactivity disorder (ADHD), narcolepsy, anxiety, generalized anxiety disorder (GAD), panic disorder, obsessive-compulsive disorder (OCD), insomnia, or pain.
